SEQUENCE LISTING (40 sequences)

SEQ ID NO: 1 - protein, 475 aa, Homo sapiens [sequence omitted]

SEQ ID NO: 2 - protein, 85 aa, Homo sapiens
MASAARLTMMWEEVTCPICLDPFVEPVSIECGHSFCQECISQVGKGGGSVCPVCRQRFLLKNLRPNRQLANMVNNLKEISQEARE

SEQ ID NO: 3 - protein, 180 aa, Homo sapiens
AVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPL

SEQ ID NO: 4 - protein, 38 aa, Homo sapiens
RCAVHGERLHLFCEKDGKALCWVCAQSRKHRDHAMVPL

SEQ ID NO: 5 - protein, 110 aa, Homo sapiens
EEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHS

SEQ ID NO: 6 - protein, 153 aa, Artificial sequence
  pOP-TS-Ube2N-WT
GMAGLPRRIIKETQRLLAEPVPGIKAEPDESNARYFHVVIAGPQDSPFEGGTFKLELFLPEEYPMAAPKVRFMTKIYHPNVDKLGRICLDILKDKWSPALQIRTVLLSIQALLSAPNPDDPLANDVAEQWKTNEAQAIETARAWTRLYAMNNI

SEQ ID NO: 7 - protein, 150 aa, Artificial sequence
  pOP-TG-Ube2V2-WT
GSQEFMAVSTGVKVPRNFRLLEELEEGQKGVGDGTVSWGLEDDEDMTLTRWTGMIIGPPRTNYENRIYSLKVECGPKYPEAPPSVRFVTKINMNGINNSSGMVDARSIPVLAKWQNSYSIKVVLQELRRLMMSKENMKLPQPPEGQTYNN

SEQ ID NO: 8 - protein, 76 aa, Artificial sequence [sequence omitted]

SEQ ID NO: 9 - protein, 1068 aa, Artificial sequence
  pET21d-Ube1
MGHHHHHHGGMSSSPLSKKRRVSGPDPKPGSNCSPAQSVLSEVPSVPTNGMAKNGSEADIDEGLYSRQLYVLGHEAMKRLQTSSVLVSGLRGLGVEIAKNIILGGVKAVTLHDQGTAQWADLSSQFYLREEDIGKNRAEVSQPRLAELNSYVPVTAYTGPLVEDFLSGFQVVVLTNTPLEDQLRVGEFCHNRGIKLVVADTRGLFGQLFCDFGEEMILTDSNGEQPLSAMVSMVTKDNPGVVTCLDEARHGFESGDFVSFSEVQGMVELNGNQPMEIKVLGPYTFSICDTSNFSDYIRGGIVSQVKVPKKISFKSLVASLAEPDFVVTDFAKFSRPAQLHIGFQALHQFCAQHGRPPRPRNEEDAAELVALAQAVNARALPAVQQNNLDEDLIRKLAYVAAGDLAPINAFIGGLAAQEVMKACSGKFMPIMQWLYFDALECLPEDKEVLTEDKCLQRQNRYDGQVAVFGSDLQEKLGKQKYFLVGAGAIGCELLKNFAMIGLGCGEGGEIIVTDMDTIEKSNLNRQFLFRPWDVTKLKSDTAAAAVRQMNPHIRVTSHQNRVGPDTERIYDDDFFQNLDGVANALDNVDARMYMDRRCVYYRKPLLESGTLGTKGNVQVVIPFLTESYSSSQDPPEKSIPICTLKNFPNAIEHTLQWARDEFEGLFKQPAENVNQYLTDPKFVERTLRLAGTQPLEVLEAVQRSLVLQRPQTWADCVTWACHHWHTQYSNNIRQLLHNFPPDQLTSSGAPFWSGPKRCPHPLTFDVNNPLHLDYVMAAANLFAQTYGLTGSQDRAAVATFLQSVQVPEFTPKSGVKIHVSDQELQSANASVDDSRLEELKATLPSPDKLPGFKMYPIDFEKDDDSNFHMDFIVAASNLRAENYDIPSADRHKSKLIAGKIIPAIATTTAAVVGLVCLELYKVVQGHRQLDSYKNGFLNLALPFFGFSEPLAAPRHQYYNQEWTLWDRFEVQGLQPNGEEMTLKQFLDYFKTEHKLEITMLSQGVSMLYSFFMPAAKLKERLDQPMTEIVSRVSKRKLGRHVRALVLELCCNDESGEDVEVPYVRYTIR

SEQ ID NO: 10 - protein, 88 aa, Artificial sequence
  pOP-TG-TRIM21-RING
GSHMASAARLTMMWEEVTCPICLDPFVEPVSIECGHSFCQECISQVGKGGGSVCPVCRQRFLLKNLRPNRQLANMVNNLKEISQEARE

SEQ ID NO: 11 - protein, 164 aa, Artificial sequence
  pOP-TG-Ubiquitin-G75/76A-TRIM21-RING
GSHMQIFVKTLTGKTITLEVEPSDTIENVKAKIQDKEGIPPDQQRLIFAGKQLEDGRTLSDYNIQKESTLHLVLRLRAAMASAARLTMMWEEVTCPICLDPFVEPVSIECGHSFCQECISQVGKGGGSVCPVCRQRFLLKNLRPNRQLANMVNNLKEISQEARE

SEQ ID NO: 12 - protein, 153 aa, Artificial sequence
  pOP-TS-Ube2N-C87K-K92A
GMAGLPRRIIKETQRLLAEPVPGIKAEPDESNARYFHVVIAGPQDSPFEGGTFKLELFLPEEYPMAAPKVRFMTKIYHPNVDKLGRIKLDILADKWSPALQIRTVLLSIQALLSAPNPDDPLANDVAEQWKTNEAQAIETARAWTRLYAMNNI

SEQ ID NO: 13 - protein, 74 aa, Artificial sequence
  pET17b-Ubiquitin-1-74
MQIFVKTLTGKTITLEVEPSDTIENVKAKIQDKEGIPPDQQRLIFAGKQLEDGRTLSDYNIQKESTLHLVLRLR

SEQ ID NO: 14 - protein, 88 aa, Artificial sequence
  pET17b-His-Ubiquitin
MGSSHHHHHHSGMQIFVKTLTGKTITLEVEPSDTIENVKAKIQDKEGIPPDQQRLIFAGKQLEDGRTLSDYNIQKESTLHLVLRLRGG

SEQ ID NO: 15 - protein, 153 aa, Artificial sequence
  pOP-TS-Ube2N-D119A
GMAGLPRRIIKETQRLLAEPVPGIKAEPDESNARYFHVVIAGPQDSPFEGGTFKLELFLPEEYPMAAPKVRFMTKIYHPNVDKLGRICLDILKDKWSPALQIRTVLLSIQALLSAPNPDAPLANDVAEQWKTNEAQAIETARAWTRLYAMNNI

SEQ ID NO: 16 - protein, 153 aa, Artificial sequence
  pOP-TS-Ube2N-D119N
GMAGLPRRIIKETQRLLAEPVPGIKAEPDESNARYFHVVIAGPQDSPFEGGTFKLELFLPEEYPMAAPKVRFMTKIYHPNVDKLGRICLDILKDKWSPALQIRTVLLSIQALLSAPNPDNPLANDVAEQWKTNEAQAIETARAWTRLYAMNNI

SEQ ID NO: 17 - protein, 153 aa, Artificial sequence
  pOP-TS-Ube2N-N79A
GMAGLPRRIIKETQRLLAEPVPGIKAEPDESNARYFHVVIAGPQDSPFEGGTFKLELFLPEEYPMAAPKVRFMTKIYHPAVDKLGRICLDILKDKWSPALQIRTVLLSIQALLSAPNPDDPLANDVAEQWKTNEAQAIETARAWTRLYAMNNI

SEQ ID NO: 18 - protein, 153 aa, Artificial sequence
  pOP-TS-Ube2N-N123A
GMAGLPRRIIKETQRLLAEPVPGIKAEPDESNARYFHVVIAGPQDSPFEGGTFKLELFLPEEYPMAAPKVRFMTKIYHPNVDKLGRICLDILKDKWSPALQIRTVLLSIQALLSAPNPDDPLAADVAEQWKTNEAQAIETARAWTRLYAMNNI

SEQ ID NO: 19 - protein, 153 aa, Artificial sequence
  pOP-TS-Ube2N-D124A
GMAGLPRRIIKETQRLLAEPVPGIKAEPDESNARYFHVVIAGPQDSPFEGGTFKLELFLPEEYPMAAPKVRFMTKIYHPNVDKLGRICLDILKDKWSPALQIRTVLLSIQALLSAPNPDDPLANAVAEQWKTNEAQAIETARAWTRLYAMNNI

SEQ ID NO: 20 - protein, 167 aa, Artificial sequence
  pET28a-Ube2D1-WT
MGSSHHHHHHSSGLVPRGSHMALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAM

SEQ ID NO: 21 - protein, 252 aa, Artificial sequence
  pOP-TG-Ubiquitin-TRIM21-RING-RING
GSHMQIFVKTLTGKTITLEVEPSDTIENVKAKIQDKEGIPPDQQRLIFAGKQLEDGRTLSDYNIQKESTLHLVLRLRAAMASAARLTMMWEEVTCPICLDPFVEPVSIECGHSFCQECISQVGKGGGSVCPVCRQRFLLKNLRPNRQLANMVNNLKEISQEAREGSHMASAARLTMMWEEVTCPICLDPFVEPVSIECGHSFCQECISQVGKGGGSVCPVCRQRFLLKNLRPNRQLANMVNNLKEISQEARE

SEQ ID NO: 22 - protein, 261 aa, Artificial sequence
  pOP-TG-Ubiquitin-TRIM21-RING-RING-FLAG
GSHMQIFVKTLTGKTITLEVEPSDTIENVKAKIQDKEGIPPDQQRLIFAGKQLEDGRTLSDYNIQKESTLHLVLRLRAAMASAARLTMMWEEVTCPICLDPFVEPVSIECGHSFCQECISQVGKGGGSVCPVCRQRFLLKNLRPNRQLANMVNNLKEISQEAREGSHMASAARLTMMWEEVTCPICLDPFVEPVSIECGHSFCQECISQVGKGGGSVCPVCRQRFLLKNLRPNRQLANMVNNLKEISQEAREGDYKDDDDK

SEQ ID NO: 23 - protein, 261 aa, Artificial sequence
  pOP-TG-Ubiquitin-TRIM21-RING-E12R-RING-E12R- FLAG
GSHMQIFVKTLTGKTITLEVEPSDTIENVKAKIQDKEGIPPDQQRLIFAGKQLEDGRTLSDYNIQKESTLHLVLRLRAAMASAARLTMMWREVTCPICLDPFVEPVSIECGHSFCQECISQVGKGGGSVCPVCRQRFLLKNLRPNRQLANMVNNLKEISQEAREGSHMASAARLTMMWREVTCPICLDPFVEPVSIECGHSFCQECISQVGKGGGSVCPVCRQRFLLKNLRPNRQLANMVNNLKEISQEAREGDYKDDDDK

SEQ ID NO: 24 - protein, 497 aa, Artificial sequence
  pGEMHE-mEGFP-Fc
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLKEFVTAAGITLGMDELYKYSDLELKLRILQSTARDPVQVKRPLLQMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALANHYTQKSLSLSPGK

SEQ ID NO: 25 - protein, 475 aa, Artificial sequence [sequence omitted]

SEQ ID NO: 26 - protein, 290 aa, Artificial sequence
  pGEMHE-TRIM21-RING-PRYSPRY
MASAARLTMMWEEVTCPICLDPFVEPVSIECGHSFCQECISQVGKGGGSVCPVCRQRFLLKNLRPNRQLANMVNNLKEISQEAREGTQGERGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 27 - protein, 375 aa, Artificial sequence
  pGEMHE-TRIM21-RING-RING-PRYSPRY
MASAARLTMMWEEVTCPICLDPFVEPVSIECGHSFCQECISQVGKGGGSVCPVCRQRFLLKNLRPNRQLANMVNNLKEISQEAREMASAARLTMMWEEVTCPICLDPFVEPVSIECGHSFCQECISQVGKGGGSVCPVCRQRFLLKNLRPNRQLANMVNNLKEISQEAREGTQGERGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 28 - protein, 560 aa, Artificial sequence
  pGEMHE-TRIM21-RING-TRIM21
MASAARLTMMWEEVTCPICLDPFVEPVSIECGHSFCQECISQVGKGGGSVCPVCRQRFLLKNLRPNRQLANMVNNLKEISQEAREMASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 29 - protein, 596 aa, Artificial sequence
  pGEMHE-mCherry-TRIM21-CC-PRYSPRY
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAQASNSLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 30 - protein, 350 aa, Artificial sequence
  pGEMHE-VhhGFP-Fc
MDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSLQMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 350 aa, Artificial sequence
  pGEMHE-VhhGFP-Fc-H433A
MDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSLQMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALANHYTQKSLSLSPGK

SEQ ID NO: 32 - protein, 454 aa, Artificial sequence
  pOP-TG-Ubiquitin-TRIM21-RING-RING-PRYSPRY
GSHMQIFVKTLTGKTITLEVEPSDTIENVKAKIQDKEGIPPDQQRLIFAGKQLEDGRTLSDYNIQKESTLHLVLRLRAAMASAARLTMMWEEVTCPICLDPFVEPVSIECGHSFCQECISQVGKGGGSVCPVCRQRFLLKNLRPNRQLANMVNNLKEISQEAREMASAARLTMMWEEVTCPICLDPFVEPVSIECGHSFCQECISQVGKGGGSVCPVCRQRFLLKNLRPNRQLANMVNNLKEISQEAREGTQGERGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 33 - protein, 369 aa, Artificial sequence
  pOP-TG-Ubiquitin-TRIM21-RING-PRYSPRY
GSHMQIFVKTLTGKTITLEVEPSDTIENVKAKIQDKEGIPPDQQRLIFAGKQLEDGRTLSDYNIQKESTLHLVLRLRAAMASAARLTMMWEEVTCPICLDPFVEPVSIECGHSFCQECISQVGKGGGSVCPVCRQRFLLKNLRPNRQLANMVNNLKEISQEAREGTQGERGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 34 - protein, 682 aa, Artificial sequence
  pOP-TG-Ubiquitin4-TRIM21-RING-RING-PRYSPRY
GSHMQIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQLEDGRTLSDYNIQKESTLHLVLRLRAAMASAARLTMMWEEVTCPICLDPFVEPVSIECGHSFCQECISQVGKGGGSVCPVCRQRFLLKNLRPNRQLANMVNNLKEISQEAREMASAARLTMMWEEVTCPICLDPFVEPVSIECGHSFCQECISQVGKGGGSVCPVCRQRFLLKNLRPNRQLANMVNNLKEISQEAREGTQGERGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 35 - protein, 606 aa, Artificial sequence
  pOP-TG-Ubiquitin3-TRIM21-RING-RING-PRYSPRY
GSHMQIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQLEDGRTLSDYNIQKESTLHLVLRLRAAMASAARLTMMWEEVTCPICLDPFVEPVSIECGHSFCQECISQVGKGGGSVCPVCRQRFLLKNLRPNRQLANMVNNLKEISQEAREMASAARLTMMWEEVTCPICLDPFVEPVSIECGHSFCQECISQVGKGGGSVCPVCRQRFLLKNLRPNRQLANMVNNLKEISQEAREGTQGERGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 36 - protein, 530 aa, Artificial sequence
  pOP-TG-Ubiquitin2-TRIM21-RING-RING-PRYSPRY
GSHMQIFVKTLTGKTITLEVEPSDTIENVKAKIQDKEGIPPDQQRLIFAGKQLEDGRTLSDYNIQKESTLHLVLRLRGGMQIFVKTLTGKTITLEVEPSDTIENVKAKIQDKEGIPPDQQRLIFAGKQLEDGRTLSDYNIQKESTLHLVLRLRAAMASAARLTMMWEEVTCPICLDPFVEPVSIECGHSFCQECISQVGKGGGSVCPVCRQRFLLKNLRPNRQLANMVNNLKEISQEAREMASAARLTMMWEEVTCPICLDPFVEPVSIECGHSFCQECISQVGKGGGSVCPVCRQRFLLKNLRPNRQLANMVNNLKEISQEAREGTQGERGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 37 - protein, 554 aa, Artificial sequence
  HLTV-Ubiquitin-TRIM21
GGSMQIFVKTLTGKTITLEVEPSDTIENVKAKIQDKEGIPPDQQRLIFAGKQLEDGRTLSDYNIQKESTLHLVLRLRAAMASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 38 - protein, 639 aa, Artificial sequence
  HLTV-Ubiquitin-TRIM21-RING-TRIM21
GGHMQIFVKTLTGKTITLEVEPSDTIENVKAKIQDKEGIPPDQQRLIFAGKQLEDGRTLSDYNIQKESTLHLVLRLRAAMASAARLTMMWEEVTCPICLDPFVEPVSIECGHSFCQECISQVGKGGGSVCPVCRQRFLLKNLRPNRQLANMVNNLKEISQEAREMASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLERSESWNLKDLDITSPELRSVCHVPGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 39 - DNA, 20 bp, Artificial sequence
  crRNA
atgctcacaggctccacgaa

SEQ ID NO: 40 - protein, 15 aa, Artificial sequence
  Linker
GTQGERGLKKMLRTC

(19) United States
(12) Patent Application Publication
(10) Pub. No.: US 2024/0132857 A1
(43) Pub. Date: Apr. 25, 2024

(54) FUSION PROTEINS COMPRISING TWO RING DOMAINS

(71) Applicant: UNITED KINGDOM RESEARCH AND INNOVATION, Swindon (GB)

(72) Inventors: Leo KISS, Swindon (GB); Dean CLIFT, Swindon (GB); Jakub LUPTAK, Swindon (GB); Leo JAMES, Swindon (GB)

(21) Appl. No.: 18/547,085

(22) PCT Filed: Feb. 22, 2022
(86) PCT No.: PCT/EP2022/054370

§ 371 Date: Aug. 18, 2023

(30) Foreign Application Priority Data
Feb. 22, 2021 (GB) 2102471.6

(51) Int. Cl. C12N 9/10; C07K 14/705; C12N 15/86

(52) U.S. Cl. CPC C12N 9/104; C07K 14/705; C12N 15/86; C07K 2319/73; C12N 2750/14143

(57) ABSTRACT

The present invention relates to fusion proteins comprising at least two RING domains and a protein targeting domain, and nucleic acid constructs encoding the same suitable for use for protein degradation in cells. The present invention also relates to compositions comprising these fusion proteins and nucleic acids, and the use of the fusion proteins and nucleic acid constructs in therapy.

### FIELD OF THE INVENTION

The present invention relates to fusion proteins and nucleic acid constructs suitable for use for protein degradation in cells. The present invention also relates to compositions comprising these fusion proteins and nucleic acids, and the use of the fusion proteins and nucleic acid constructs in therapy.

### BACKGROUND OF THE INVENTION

Protein degradation occurs naturally within cells and provides an endogenous mechanism to prevent the occurrence of misfolded proteins, and to mediate cellular responses. The major pathway for protein degradation is via the ubiquitin-proteasome system (UPS). The ability to manipulate the UPS in order to redirect the system and provide targeted protein degradation within cells has enormous potential for applications in research, drug discovery and therapeutics.

Selective depletion of a target protein enables the study of protein function and dynamic protein interactions at the cellular level. Such selective depletion is of particular use in drug discovery, where small molecules known as “proteolysis-targeting chimeras” (PROTACs) can be used to redirect protein degradation to induce selective depletion of a target protein (Schapira et al. (2019) Nature Reviews Drug Discovery, 18:949-963). Similarly, technologies such as “Trim-Away” utilise a specific component of the UPS, an E3 ubiquitin ligase known as TRIM21, to selectively deplete antibody-bound target proteins (Clift et al. (2017) Cell, 172:1692-1706; Zeng et al. (2020) Available as a pre-print from bioRxiv, doi: https://doi.org/10.1101/2020.07.28.225359 (and now published as Zeng et al (2021) Natural Structural & Molecular Biology vol 28, 278-289); Castro-Dopico, T., et al. (2019). Immunity 50, 1099-1114 e1010; Chen, X et al. (2019). Genome Biology 20, 19). These emerging tools and drug discovery platforms enable the study of protein interactions in a post-translational setting, and avoid many limitations associated with genetic manipulation, which can fail to provide phenotypic insight and can be costly and time-consuming.

Furthermore, targeted protein degradation holds potential for use in therapeutic applications (Wu, T, et al. (2020) Nature Structural & Molecular Biology, 27:605-614), in particular for use in diseases associated with excessive protein production or aberrant protein aggregation. The use of targeted protein degradation as a therapeutic strategy could minimise the off-target effects of drugs and avoid or reduce systemic drug exposure.

Despite the potential for harnessing the endogenous cellular protein degradation machinery, reducing this theoretical approach to practice has proved challenging. Much of the UPS, including key enzymes such as the E3 ubiquitin ligases, remains uncharacterised, and existing tools have significant limitations that make them unsuitable for practical use. For example, PROTACs can be of low potency and may require high concentrations to induce sufficient degradation (Buckley et al. Angew Chem. Int. Ed. Engl. 53:2312-30 (2014)). The identification of suitable binders for use as PROTACs that enable recruitment of E3 ubiquitin ligases and bind the target protein for degradation is also a challenge (Chopra, Sadok and Collins (2019) Drug Discov Today Technol, 31:5-13). Trim-Away based approaches are also limited in that they are unsuitable for degradation of monomeric proteins and small oligomers.

Consequently, there is a need for further fusion proteins and corresponding nucleic acid constructs which can be used to selectively degrade proteins in cells. Such fusion proteins would be useful in particular in both therapeutic and research settings.

### SUMMARY OF THE INVENTION

The present invention is directed to fusion proteins and nucleic acid constructs that encode such proteins, suitable for degrading proteins in cells. Specifically, the fusion proteins comprise two RING domains and a protein targeting domain.

In a first aspect, the present invention provides a fusion protein comprising:
a first RING domain;
a second RING domain; and
a protein targeting domain.

The first RING domain and second RING domain are capable of dimerization. The invention provides fusion constructs having E3 ubiquitin ligase activity.

The inventors have shown that by providing a construct comprising at least two RING domains capable of dimerization, when the fusion protein is in close proximity to another fusion protein also comprising two RING domains, sufficient self-ubiquitination can occur to enable efficient protein degradation. The two RING domains of each fusion protein dimerise and when the RING dimers of each fusion protein are in close proximity, for example co-localised on a Fc, oligomeric protein or proteins with short sequence repeats, one RING dimer is available to mediate ubiquitination of the other. The RING domains having self-ubiquitination activity. Therefore, the fusion constructs are capable of self-ubiquitination.

The protein targeting domain can be positioned relative to the first and second RING domain such that when co-localised on a target protein with a second fusion protein the distance between the RING dimer formed by the first and second RING domains of the first fusion protein and the RING dimer formed by the first and second RING domains of the first fusion protein is in the range of 8-10 nm, preferably approximately 9 nm.

The separate domains of the fusion protein may be provided in the order of RING Domain-RING Domain-Protein Targeting domain. In one embodiment the protein targeting domain can be located at the C-terminal end of the first and second RING domains.

In one embodiment the fusion protein does not comprise a coiled-coil domain and/or a B-box domain. In a further embodiment the fusion protein does not comprise a coiled-coil domain and a B-box domain. In one embodiment the fusion protein does comprise a B-Box, but preferably does not comprise a coiled-coil domain, for example the fusion protein may comprise a B-Box domain between the RING domains and protein targeting domain.

In one embodiment the first RING domain and second RING domain are derived from TRIM polypeptides. The TRIM polypeptides may be selected from the group comprising but not limited to TRIM5, TRIM7, TRIM19, TRIM21, TRIM25, TRIM28 and TRIM32. The first RING domain and the second RING domain can be derived from the same TRIM polypeptide. Preferably the first RING domain and second RING domain are derived from a TRIM21 polypeptide. Preferably the fusion protein comprises two RING domains.

In one embodiment the protein targeting domain is a PRYSPRY domain. In another embodiment the protein targeting domain is an antibody, antibody fragment thereof, or antibody mimetic. Preferably the antibody fragment is selected from the group consisting of a Fab, Fab′, F(ab′)2, scFab, Fv, scFV, dAB, VL fragments thereof, VH fragment thereof and sdAb (i.e. nanobodies) such as VHH fragments thereof. More preferably the protein targeting domain is a scFV or VHH.

The fusion protein can comprise linker sequences between each of the domains. The fusion protein can comprise a linker sequence between the first and second RING domains and/or a linker sequence between the second RING domain and the protein targeting sequence.

A second aspect of the invention provides a nucleic acid construct that encodes the fusion protein of the first aspect of the invention.

A third aspect of the invention provides a nucleic acid construct comprising a first nucleic acid sequence encoding a first RING domain, a second nucleic acid sequence encoding a second RING domain, and a third nucleic acid sequence encoding a protein targeting domain.

In one embodiment the nucleic acid does not encode for a coiled-coil domain, does not encode for a B-Box domain or does not encode for a coiled-coil domain and a B-box domain. In one embodiment the nucleic acid further encodes for a B-Box domain, but preferably does not encode for a coiled-coil domain.

The nucleic acid constructs of the second and third aspects of the invention, may be in the form of a vector. The vector can be a viral or non-viral delivery vector, preferably a viral delivery vector including adeno-associated virus (AVV) vector or a lentivirus vector.

A fourth aspect of the invention provides a pharmaceutical composition comprising a fusion protein or a nucleic acid construct of the first, second, or third aspects of the invention. The pharmaceutical composition may further comprise a pharmaceutically acceptable carrier and/or excipient.

A fifth aspect of the invention provides a method of treating a neurological disorder, a viral infection or a trinucleotide repeat disorder, the method comprising administering a fusion protein or a nucleic acid of the first, second, or third aspects of the invention, or the pharmaceutical composition of the fourth aspect of the invention to a subject.

The neurological disorder to be treated may be Alzheimer's Disease or Huntington's Disease. The infection to be treated may be a viral infection, for example an HIV infection. Trinucleotide repeat disorders include but are not limited to Huntington's disease, Dentatorubropallidoluysian atrophy and spinocerebellar ataxia.

The method may further comprise administering, simultaneously or sequentially, in any order, an antibody or antibody fragment thereof, or a nucleic acid construct encoding the antibody or antibody fragment thereof.

A seventh aspect of the invention provides a fusion protein or a nucleic acid of first, second, or third aspects of the invention for use as a medicament.

In one embodiment the fusion protein or nucleic acid may be for use in the treatment of a neurological disorder. The neurological disorder may be a disorder such is Alzheimer's Disease or Huntington's Disease.

In one embodiment the fusion protein or a nucleic acid may be for use in the treatment of an infection. The infection may be a viral infection, such as an HIV infection.

In one embodiment the fusion protein or a nucleic acid may be for use in the treatment of a trinucleotide repeat disorder. Trinucleotide repeat disorders include but are not limited to Huntington's disease, Dentatorubropallidoluysian atrophy and spinocerebellar ataxia.

An eight aspect of the invention provides the use of the fusion protein or the nucleic acid of the first, second, or third aspects of the invention in the manufacture of a medicament. The medicament may be for use in the treatment of a neurological disorder, an infection or a trinucleotide repeat disorder, as described above.

A ninth aspect of the invention provides a method of degrading a protein in a cell comprising introducing a fusion protein or the nucleic acid of the first, second, or third aspects of the invention into a cell. In one embodiment the cell is an in vitro cell. The method may further comprise introducing the protein or nucleic acid into the cell by transfection or transduction, preferably by using a vector, electroporation or injection.

The method may further comprise introducing an antibody or antibody fragment thereof or a nucleic acid encoding the antibody or fragment thereof into the cell.

A tenth aspect of the invention provides a method of degrading a protein in a sample comprising introducing a fusion protein or nucleic acid of the first, second, or third aspects of the invention into a sample. The method may further comprise introducing the protein or nucleic acid into the sample by transfection or transduction, preferably by using a vector, electroporation or injection.

The method may further comprise introducing an antibody or antibody fragment thereof or a nucleic acid encoding the antibody or fragment thereof or a nucleic acid encoding the same into the cell or sample into the sample.

All preferred features of the second and subsequent aspects of the invention are as for the first aspect mutatis mutandis.

### BRIEF DESCRIPTION OF FIGURES

FIG. 1: Structure of initiation of RING-anchored ubiquitin chain elongation. a) Side and top view of the Ub-R:Ube2N˜Ub:Ube2V2 structure (Ub-R, Ub in red, R in blue, Ube2N˜Ub, Ube2N in green, Ub in orange, Ube2V2 in teal). Chains drawn as cartoon represent the asymmetric unit. b) The canonical model of initiation of RING-anchored ubiquitin chain elongation. c) Schematic cartoon, representing the canonical model of RING-anchored ubiquitin chain elongation shown in b). Symmetry mates are denoted by ′ next to the label. Ub, ubiquitin, Ub-R, ubiquitin-RING.

FIG. 2: Chemical mechanism of ubiquitination. a) Magnified regions of the active site of Ube2N˜Ub/Ube2V2 (Ube2N in green, donor Ub in orange, acceptor Ub in red, Ube2V2 in teal). b) Chemical scheme for the activation of the acceptor lysine. c) Acid coefficients (pKₐ), d) K_{M} and e) k_{cat} of di-ubiquitin formation by Ube2N/V2 are presented as best fit+standard error. Ub, ubiquitin.

FIG. 3: The mechanism of RING-anchored ubiquitination in trans. a) Surface representation of the canonical model of the Ub-R:Ube2N˜Ub:Ube2V2 structure (Ub-R, Ub in red, R in blue, Ube2N˜Ub, Ube2N in green, Ub in orange, Ube2V2 in teal). b) Domain architecture of TRIM21 constructs used in biochemical assays. c) Cartoon models of substrate (Fc, gray) engagement by TRIM21 constructs (blue). d) Substrate (Fc) induced self-ubiquitination assay of 100 nM Ub-TRIM21 constructs. Reactions were incubated for 5 min at 37° C. *(asterisk) indicates a TRIM21 degradation product that could not be removed during purification. Western blots are representative of n=3 independently performed experiments. Uncropped blots are provided in Source Data. Ub, ubiquitin; R, RING; B, Box; CC, coiled-coil; PS, PRYSPRY; kDa, kilo Dalton.

FIG. 4: The mechanism of RING-anchored ubiquitination in cis. a) For ubiquitination in cis, the RING-anchored (blue) ubiquitin chain (red) must be sufficiently long to reach the active site on Ube2N˜Ub/Ube2V2 (Ube2N in green, Ub in orange, Ube2V2 in teal). The chain can go around two different routes, one shown here and the other not shown The ubiquitin chain was modelled using the Ub-R:Ube2N˜Ub:Ube2V2 structure and a K63-linked Ub₂ structure (2JF5³⁴) using PyMol. b) Substrate (Fc) induced self-ubiquitination assay of 100 nM Ubₙ-TRIM21 constructs. Reactions were incubated for 5 min at 37° C. Western blots are representative of n=3 independently performed experiments. Uncropped blots are provided in Source Data. Ub, ubiquitin; R, RING; PS, PRYSPRY; kDa, kilo Dalton.

FIG. 5: Catalytic RING topology drives targeted protein degradation. a) Schematic cartoon showing the topology of TRIM21 (blue) on GFP-Fc (green and gray, respectively). b), c) GFP-Fc degradation assay. b) Western blot of RPE-1 TRIM21-knock-out cells transiently expression GFP-Fc and a series of TRIM21 constructs. Western blots are representative of n=2 independently performed experiments. c) Shown is the flow cytometry analysis of green fluorescence of RPE-1 TRIM21-knock-out cells transiently expressing GFP-Fc and a series of TRIM21 constructs. After electroporation, each population of cells was split in two and either treated with MG132 or DMSO. Data are presented as mean±standard error of the mean. Each data point in the graph represents one biologically independently performed experiment (n=3 (for mCh-CC-PS, R-R-B-CC-PS, R-PS) or 4 (R-B-C-C-PS, R-R-PS)). A two-tailed unpaired student T test was performed to assess the significance of fluorescence reduction relative to mCh-CC-PRYSPRY (P values: R-B-CC-PRYSPRY, 0.0797 (ns); R-R-B-CC-PRYSPRY, 0.02366 (ns); R-PRYSPRY, 0.4964 (ns); R-R-PRYSPRY, 0.0035 (**)). d, e) Trim-Away of Caveolin-1-mEGFP (Cav1-GFP) in NIH 3T3 GFP-Cav-1-knock in cells. Shown in d) is the normalized GFP fluorescence (error bars represent ±SEM of 4 images) and in e) the western blot after the experiment. Data in d, e) are representative of n=2 independent experiments. Uncropped blots and raw data are provided in Source Data. R, RING; B, Box; CC, coiled-coil; PS, PRYSPRY; mCh, mCherry; kDa, kilo Dalton; ns, not significant.

FIG. 6: TRIM protein assembly on viruses. Cartoon models of the assembly of TRIM5 (a, b) and TRIM21 (c, d) on viral capsids. a) Shown is the hexagonal assembly of TRIM5 on HIV-1 capsid as imaged by cryo-electron tomography. c) Assembly of TRIM21:antibody complexes on adenovirus capsid (adenoviral measurements are based on 6B1T). b, d) Cartoons visualizing how the TRIM protein assembly on the viral capsid enables formation of the catalytic RING topology. R, RING; B, Box; CC, coiled-coil; PS, PRYSPRY; Ub, ubiquitin.

FIG. 7: Structural models for distances of different TRIM21 constructs. a) Domain architecture of TRIM21 constructs used in biochemical and cellular assays. For biochemical experiments, the N-terminus of TRIM21 was mono-ubiquitinated. b) Structure of TRIM21 PRYSPRY (blue) in complex with Fc (gray, 2IWG). The distance shown spans from the N-terminal His of one to the other. c) Structure of TRIM5a-B-Box-coiled-coil (blue, 4TN3). TRIM21 and TRIM5a coiled-coils align well by sequence and show no insertions. Thus, TRIM5a-coiled-coil is a suitable model for the corresponding region of TRIM21. The distance shown spans from the N-terminus of one B-box to the other. d) Structural model of Ub-R-R-PRYSPRY:Fc during initiation of ubiquitin chain elongation. Our UbR:Ube2N˜Ub:Ube2V2 (7BBD, Ub-R, Ub in red and R in blue; Ube2N˜Ub, Ube2N in green and Ub in orange; Ube2V2 in teal) structure (as the canonical model) was superposed on the TRIM21-PRYSPRY:Fc structure. Lines indicate the linkers between RING and PRYSPRY. Ub, ubiquitin; R, RING.

FIG. 8: Ube2D1 cannot mediate TRIM21 ubiquitination via the catalytic RING topology. Fc-induced self-ubiquitination assay of 100 nM Ub-TRIM21 in the presence of 0.5 μM Ube2D1. Western blots represent n=2 independently performed experiments. Ub, ubiquitin; R, RING; B, Box; CC, coiled-coil; PS, PRYSPRY; kDa, kilo Dalton.

FIG. 9: The theoretical structure of a fusion protein construct according to the invention comprising a first RING domain (R1) positioned at the N-terminal end of the second RING domain (R2), and the protein targeting domain (PT) position at the C-terminal end of the second RING domain.

FIG. 10: TRIM21 constructs for improved targeted protein degradation. a) Catalysis of unanchored ubiquitin chains by Ube2N/Ube2V2 of different TRIM21 constructs at 10 μM concentration. Shown is an InstantBlue gel of the reactions after 60 min. b) Trim-Away of endogenous IKKα in RPE1 TRIM21 knock-out cells using transiently expressed TRIM21 constructs. c) Trim-Away of endogenous Erk1 kinase in either RPE1 WT or TRIM21 knock-out cells using R-R-PS protein at 2 μM concentration in the electroporation reaction. Endogenous TRIM21 in RPE-1 cells would usually take 3-4 h for efficient Trim-Away of Erk1. d) Trim-Away of ectopically expressed monomeric EGFP in RPE1 cells using mono- or poly-clonal antibody against GFP and different TRIM21 constructs. Shown is the relative GFP intensity after 4.5 h. T21, TRIM21; R, RING; B, Box; CC, coiled-coil; PS, PRYSPRY.

FIG. 11: Targeted protein degradation. a) Degradation of Caveolin-1-EGFP monitored using real-time fluorescence microscopy. b) Western blot showing levels of the constructs at the end of the assay. c) Western blot showing levels of the anti-GFP antibody at the end of the assay. T21, TRIM21; T21R, TRIM21 RING; PS, PRYSPRY.

FIG. 12: Targeted protein degradation. Degradation of H2B-EGFP monitored using real-time fluorescence microscopy. Ub, ubiquitin; T21R, TRIM21 RING.

### DETAILED DESCRIPTION OF INVENTION

The inventors have found that a fusion protein comprising at least two RING domains and a Protein Targeting Domain is capable of forming part of a catalytic RING topology that enables protein degradation of the target protein. Co-localisation of two fusion proteins comprising this structure can induce a specific RING topology, that enables self-ubiquitination and subsequent protein degradation. The RING dimer of the fusion protein acts as an enzyme, having E3 ubiquitin ligase activity, and at least one more RING domain (for example from a co-localised second fusion protein) acts as a substrate in the reaction for ubiquitin chain formation. The three RING domains form a catalytic RING topology that enables protein degradation of the target protein. This topology is required for the use of the heterodimeric E2 enzyme Ube2N/Ube2V2 to form the ubiquitin chain on TRIM21.

The fusion protein can be used to target a wider selection of targets than if a full-length TRIM polypeptide was be used, in particular if a TRIM21 polypeptide was used. In particular the fusion protein can be used to target monomeric proteins and small oligomers, including dimeric proteins. Protein targets include but are not limited to kinases, transcription factors or other disease-causing proteins. Furthermore, the fusion protein of the invention is easier to produce than constructs using the full length TRIM21 polypeptide comprising only one RING domain. The fusion proteins may also have higher activity and may act faster in protein depletion and more efficiently than the use of wildtype TRIM21. The fusion proteins comprising two RING domains may be more efficient degraders of target protein than corresponding fusion proteins comprising only one RING domain.

The fusion protein and the target protein, and optionally an antibody to the target protein, form a complex enabling degradation of the complex and depletion of the protein.

Accordingly, the invention provides a fusion protein comprising:
a first RING domain;
a second RING domain; and
a protein targeting domain.

The first RING domain and second RING domain are capable of dimerization. The fusion proteins of the invention have E3 ubiquitin ligase activity. The RING domains can have self-ubiquitination activity. The first and second RING domains and the protein targeting domain are arranged such that when the fusion protein is co-localised with a third RING domain, a catalytic RING topology can be obtained.

By a “catalytic RING topology” it is meant a structure resulting in an approximately 9 nm separation between the enzyme RING and the substrate RING, in which a RING dimer acts as an enzyme and at least one further RING acts as the substrate for ubiquitination.

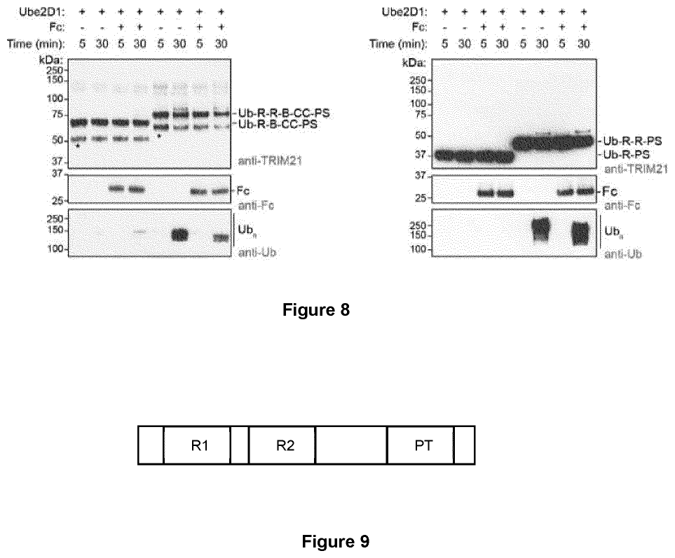

Linker sequences may be provided between each domain. In one embodiment the protein targeting domain is at the C-terminal end of the first and second RING domains. The separate domains of the fusion protein can be provided in the order of RING Domain-RING Domain-Protein Targeting Domain as shown in FIG. 9 in which the first RING domain is provided at the N-terminal end of the second RING domain and the protein targeting domain is provided at the C-terminal end of the second RING domain. In such an embodiment the amino acid sequence of the first RING domain is linked to the N-terminal of the second RING domain and the protein targeting domain is linked to the C-terminal domain of the second RING domain. A fusion protein according to the invention may have additional N-terminal and/or C-terminal amino acid sequences, and/or additional domains located between the RING domains and protein targeting domain.

The RING domains of the fusion protein may be derived from any suitable polypeptide. RING domains are known in the art and were described in Freemont P S et al (1991) A novel cysteine-rich sequence motif. Cell 64: 483-484 and function as E3 ligases (Meroni G and Roux G, TRIM/RBCC, a novel class of ‘single protein RING finger’ E3 ubiquitin ligases (2005) BioEssays 27, 11:1147-1157).

The RING domains used in the fusion proteins of the invention have E3 ubiquitin ligase activity. The RING domain of TRIM21 is an E3 ubiquitin ligase and targets ubiquitin conjugating enzymes to the substrate. Members of the RING (Really Interesting New Gene) domain family typically have the consensus sequence Cys-X₂-Cys-X(₉₋₃₉)-Cys-X(₁₋₃)-His-X(₂₋₃)-(Ans/Cys/His)-X₂-Cys-X(₄₋₄₈)-Cys-X₂-Cys (Deshaies R J et al and Joazeiro C et al, RING Domain E3 Ubiquitin Ligases, Annu. Rev. Biochem (2009) 78:399-434). RING E3 ligase domains are found in a variety of proteins. Other RING domains include a RING domain from a protein X-linked mammalian inhibitor of apoptosis (XIAP) and a RING domain of DER3/Hrd1. Therefore, the use of RING domains derived from other protein families in the fusion proteins are also encompassed. The RING domains may be capable of self-ubiquitination, i.e. have self-ubiquitination activity.

Preferably the RING domains of the fusion protein are derived from a TRIM polypeptide. The TRIM family comprise a large number of RING E3 ligases (Marin, I. Origin and diversification of TRIM ubiquitin ligases. PLoS One 7, e50030 (2012)). In a preferred embodiment the RING domain is derived from a TRIM21 polypeptide, preferably human TRIM21. The sequence of human TRIM21 is set forth in SEQ ID NO: 1 (Uniprot: P19474).

(SEQ ID NO: 1)
MASAARLTMMWEEVTCPICLDPFVEPVSIECGHSFCQECISQVGKGGGS
VCPVCRQRFLLKNLRPNRQLANMVNNLKEISQEAREGTQGERCAVHGER
LHLFCEKDGKALCWVCAQSRKHRDHAMVPLEEAAQEYQEKLQVALGELR
RKQELAEKLEVEIAIKRADWKKTVETQKSRIHAEFVQQKNFLVEEEQRQ
LQELEKDEREQLRILGEKEAKLAQQSQALQELISELDRRCHSSALELLQ
EVIIVLERSESWNLKDLDITSPELRSVCHVPGLKKMLRTCAVHITLDPD
TANPWLILSEDRRQVRLGDTQQSIPGNEERFDSYPMVLGAQHFHSGKHY
WEVDVTGKEAWDLGVCRDSVRRKGHFLLSSKSGFWTIWLWNKQKYEAGT
YPQTPLHLQVPPCQVGIFLDYEAGMVSFYNITDHGSLIYSFSECAFTGP
LRPFFSPGENDGGKNTAPLTLCPLNIGSQGSTDY

The RING domain of human TRIM21 comprises at least amino acids 3-81 of human TRIM21 sequence as set forth in SEQ ID NO: 1, preferably amino acids 1 to 85 of human TRIM21 amino acid sequence as set forth in SEQ ID NO: 1. The RING domain comprising amino acid 1 to 85 of human TRIM21 comprises the sequence:

(SEQ ID NO: 2)
MASAARLTMMWEEVTCPICLDPFVEPVSIECGHSFCQECISQVGKGGGS
VCPVCRQRFLLKNLRPNRQLANMVNNLKEISQEARE

Therefore, in one embodiment of the invention the RING domains comprise amino acids 3-81 of SEQ ID NO: 2, preferably amino acid residues 1-81 of SEQ ID NO: 2, more preferably the sequence of SEQ ID NO: 2 or a variant thereof. Preferably the variant sequence has at least 60% identity to the reference sequence, using the default parameters of the BLAST computer program (Atschul et al., J. Mol. Biol. 215, 403-410 (1990) provided by HGMP (Human Genome Mapping Project), at the amino acid level. More preferably, the variant sequence of SEQ ID NO: 2 may have at least 65%, 70%, 75%, 80%, 85%, 90% and still more preferably 95% (still more preferably at least 99%) identity, at the amino acid level, to the sequence of SEQ ID NO:2.

“Identity” as known in the art is the relationship between two or more polypeptide sequences or two or more polynucleotide sequences, as determined by comparing the sequences. In the art, identity also means the degree of sequence relatedness (homology) between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic acids Research, 12, 387 (1984), BLASTP, BLASTN, and FASTA (Atschul et al., J. Molec. Biol. 215, 403 (1990).

In some embodiments RING domains from a TRIM polypeptide other than TRIM21 can be used, for example a RING domain from TRIM5, TRIM7, TRIM 19, TRIM25, TRIM28 and/or TRIM32, preferably a RING domain from TRIM5 may be used.

The fusion protein comprises at least two RING domains, i.e. 2, 3 or more domains, preferably the fusion comprises 2 or 3 RING domains, more preferably 2 RING domains. The RING domains have sequences capable of dimerizing with each other to form a RING dimer. Preferably the RING domains comprise the same sequence. In one embodiment the first RING domain and second RING domain both comprise the sequence of SEQ ID NO: 2. If the RING domains comprise different sequences, at least the sequences of the first and second RING domains should be capable of dimerizing with each other to form a RING dimer. In one embodiment the first RING domain comprises the sequence of SEQ ID NO: 2 and the second RING domain comprises a variant sequence of SEQ ID NO: 2, or vice versa. The variant sequence may have at least 65%, 70%, 75%, 80%, 85%, 90% and preferably 95% (still more preferably at least 99%) identity, to the sequence of SEQ ID NO:2.

The protein targeting domain directs the fusion protein to the target protein to be degraded, also referred to as a protein of interest. The protein targeting domain, binds the target protein or antibody or fragment thereof or antibody mimetic binding the same, and may also be referred to as a “protein binding domain”. The protein targeting domain may either bind the target protein directly to form a Fusion protein-Target protein complex, or bind to an antibody, antibody fragment thereof or antibody mimetic binding the target protein to form a Fusion protein-Antibody-Target protein complex. The protein targeting domain is preferably connected to the C-terminal end of the two RING domains.

In one embodiment the protein targeting domain is the PRYSPRY domain. In such an embodiment the fusion protein comprises a first RING domain; a second RING domain; and a PRYSPRY domain. The PRYSPRY domain preferably being located at the C-terminal end of the first and second RING domains.

In one embodiment when the protein targeting domain is the PRYSPRY domain the fusion protein comprises a first RING domain; a second RING domain; and a PRYSPRY domain, wherein the protein does not comprise a coiled-coil domain or a B-box domain. The PRYSPRY domain preferably being located at the C-terminal end of the first and second RING domains.

In one preferred embodiment the fusion protein comprises a first RING domain; a second RING domain; and PRYSPRY domain at the C-terminal end of the first and second RING domain, wherein the RING domains are derived from a TRIM polypeptide, preferably TRIM21. Preferably the fusion protein does not comprise a coiled-coil domain and/or a B-box domain derived from TRIM located between the PRYSPRY domain and the second RING domain, more preferably the fusion protein does not comprise any coiled-coil domain or B-box domain sequence.

The PRYSPRY domain can be derived from a TRIM polypeptide preferably TRIM21, more preferably human TRIM21. The PRYSPRY domain is comprised of the PRY and SPRY regions at positions 286-337 and 339-465 of the human TRIM21 amino acid sequence as set forth in SEQ ID NO: 1.

Preferably the PRYSPRY domain comprises the sequence:

(SEQ ID NO: 3)
AVHITLDPDTANPWLILSEDRRQVRLGDTQQSIPGNEERFDSYPMVLGA
QHFHSGKHYWEVDVTGKEAWDLGVCRDSVRRKGHFLLSSKSGFWTIWLW
NKQKYEAGTYPQTPLHLQVPPCQVGIFLDYEAGMVSFYNITDHGSLIYS
FSECAFTGPLRPFFSPGENDGGKNTAPLTLCPL

In one embodiment of the invention, the PRYSPRY domain comprises the sequence of SEQ ID NO: 3 or a variant thereof. Preferably the variant sequence has at least 60% identity to the reference sequence, using the default parameters of the BLAST computer program (Atschul et al., J. Mol. Biol. 215, 403-410 (1990) provided by HGMP (Human Genome Mapping Project), at the amino acid level. More preferably, the variant sequence of SEQ ID NO: 3 may have at least 65%, 70%, 75%, 80%, 85%, 90% and preferably 95% (still more preferably at least 99%) identity, at the amino acid level, to the sequence of SEQ ID NO:3.

The PRYSPRY domain of the fusion proteins binds to the Fc of an antibody or antibody fragment thereof, for example the Fc region of a human IgG1. The fusion protein binds the antibody bound to the target protein.

The Fc is a dimer and therefore can be bound by two PRYSPRY domains. The PRYSPRY domain of a first fusion protein binds one of the monomers of the Fc, whilst the PRYSPRY domain of a second fusion protein binds the second monomer of the Fc. This co-localises two fusion proteins bringing the RING dimers of each fusion protein into close proximity, so that one RING dimer of one fusion protein is available to mediate the ubiquitination of the other RING dimer.

In a further embodiment of the invention the protein targeting domain is an antibody, antibody fragment thereof, or antibody mimetic. Preferably the antibody fragment molecule is selected from the group consisting of a Fab, Fab′, F(ab′)2, scFab, Fv, scFV, dAB, VL fragments thereof, VH fragments thereof and sdAb (i.e. nanobodies) such as VHH fragments thereof. Preferably an scFV or VHH.

In one preferred embodiment the fusion protein comprises a first RING domain; a second RING domain; and a VHH domain, wherein the RING domains are derived from a TRIM polypeptide, preferably TRIM21, wherein the VHH binds to a protein of interest, preferably wherein the VHH is at the C-terminal end of the first and second RING domains. Preferably the fusion protein does not comprise a coiled-coil domain and/or a B-box domain derived from TRIM located between the VVH domain and the second RING domain, more preferably the fusion protein does not comprise any coiled-coil domain or B-box domain sequence.

The antibody, antibody fragment thereof or antibody mimetic of the fusion protein specifically binds to the target protein. The fusion protein directly binds the target protein to be degraded at a target sequence of the target protein. Many proteins are oligomeric (or at least dimers) or part of a protein complex, therefore the antibody domain of a first fusion protein can bind one of the monomers of the oligomer or protein complex, whilst the antibody domain of a second fusion protein binds a second monomer of the oligomer or protein complex. This co-localises two fusion proteins bringing the RING dimers of each fusion protein into close proximity, so that one RING dimer of one fusion protein is available to mediate the ubiquitination of the other RING dimer.

In one embodiment the target protein can be a protein having a pathogenic form and a non-pathogenic form. The protein targeting domain binds the pathogenic form but does not bind the non-pathogenic form of the protein. The pathogenic form of the target protein may comprise a repeat domain or is a multimeric form of the protein.

The target protein may be an intracellular protein selected from the group comprising of huntingtin and tau. If the intracellular protein is huntingtin, in one embodiment the protein target domain of the fusion protein binds to a poly-glutamate sequence of huntingtin.

Preferably the fusion protein does not comprise a B-box domain and a coiled-coil domain of TRIM21 located between the second RING domain the protein targeting domain. The fusion protein may not comprise a B-box domain and a coiled-coil domain derived from any protein located between the second RING domain the protein targeting domain. In one embodiment the fusion protein does not comprise a B-box domain, such as a B-box domain derived from TRIM21, preferably does not comprise a B-box domain derived from any protein. In one embodiment the fusion does not comprise a coiled-coil domain derived from TRIM21, preferably does not comprise a coiled-coil domain derived from any protein.

The B-box domain of human TRIM21 comprises amino acid 91 to 128 of the human TRIM21 amino acid sequence as set forth in SEQ ID NO: 1. The coiled-coil domain of human TRIM21 comprises amino acids 128 to 238 of the human TRIM21 amino acid sequence as set forth in SEQ ID NO: 1.

The B-box domain can comprise the sequence

(SEQ ID NO: 4)
RCAVHGERLHLFCEKDGKALCWVCAQSRKHRDHAMVPL

Therefore, in one embodiment the fusion protein does not comprise the sequence of SEQ ID NO: 4 or a variant thereof.

The coiled coil domain can comprise the sequence:

(SEQ ID NO: 5)
EEAAQEYQEKLQVALGELRRKQELAEKLEVEIAIKRADWKKTVETQKSR
IHAEFVQQKNFLVEEEQRQLQELEKDEREQLRILGEKEAKLAQQSQALQ
ELISELDRRCHS

Therefore, in one embodiment the fusion protein does not comprise the sequence of SEQ ID NO: 5 or a variant thereof.

Preferably the fusion protein does not comprise the sequence of SEQ ID NO: 4 and SEQ ID NO: 5 or functional variants thereof. Preferably the variant sequence has at least 60% identity to the reference sequence, using the default parameters of the BLAST computer program (Atschul et al., J. Mol. Biol. 215, 403-410 (1990) provided by HGMP (Human Genome Mapping Project), at the amino acid level. More preferably, the variant sequence of SEQ ID NO: 4 or 5 may have at least 65%, 70%, 75%, 80%, 85%, 90% and preferably 95% (still more preferably at least 99%) identity, at the amino acid level, to the sequence of SEQ ID NO:4 or 5.

By not including the coiled-coil domain and the B-box domain between the second RING domain and the protein targeting domain assists in allowing the RING dimers of a fusion protein to be in close proximity with the RING dimers of a second fusion protein co-localised on the target protein (or antibody binding the target protein). This means that one RING dimer of one fusion protein is available to mediate the ubiquitination of the other RING dimer.

However, in some embodiment the fusion construct may comprise a coiled-coil domain, a B-box domain, or a coiled-coil domain and a B-box domain. If a coiled-coil domain and/or a B-box are present in the fusion protein, they should be located at a sufficient distance from the protein targeting domain and RING domains such that the RING dimer of a first fusion protein can still be in close proximity to the RING dimer of a second fusion protein, co-localised on the target protein (or antibody binding the target protein), for example when both are bound to the same Fc, to enable the RING dimers to mediate ubiquitination between each other.

The two RING domains and the protein targeting domain can be separated by linker sequences. The linker sequences may be derived from a sequence of a TRIM polypeptide, wherein the linker sequence does not encode for the coiled-coil domain and/or the B-box domain of a TRIM polypeptide.

In other embodiments standard linker sequence known in the art may be also be used, for example polyglycine or polyserine amino acid sequences may be used. The linker length can vary in size. However, the linker sequence between the two RING domains should be of sufficient length to provide flexibility to the fusion protein and enable dimerization of the two RING domains present. In one embodiment the linker sequence between the protein targeting domain and the RING domain is between 1-50 amino acid in length, preferably 1-35, 1-30, 1-25, 1-20, 1-15 or 1-10 amino acids in length. More preferably the linker is 1-6 amino acids in length, for example 1, 2, 3, 4, 5, or 6 amino acids in length. In some embodiments no linker may be present between the first and second RING domains.

The linker sequence between the RING domain and the protein targeting domain, should of be a length sufficient that enables the RING dimer of first fusion protein to be in close proximity to the RING dimer of a second fusion protein when co-localised on the target protein (or antibody binding the target protein). The linker should be of sufficient length to enable formation of the catalytic RING topology with a RING domain of a second protein.

For example, when the protein targeting domain is a PRYSPRY domain, the linker between the PRYSPRY domain and the RING domain should be of length that when bound to an Fc, the RING dimer of a first fusion protein is in close proximity to the RING dimer of a second fusion protein also bound to the Fc. Preferably the two RING dimers are located with approximately 8-10 nm of each other preferably, 9 nm within each other when bound to an Fc.

The separation of the two dimers can be determined as set out in the examples, for example using X-ray crystallography to determine the structure of the complexes and measuring the distance between the RINGs in this structure. The distance is the space between the enzyme RING of the first fusion protein and the substrate RING of the second fusion protein.

In one embodiment the linker sequence between the protein targeting domain and the RING domain is between 5 and 50 amino acid in length, preferably 5-40, 5-30, 5-25, 10-25, 15-25, 15-20 or 10-20 amino acids in length. More preferably the linker is between 10-20 amino acids in length.

In one embodiment the linker sequences may be derived from a sequence of a TRIM polypeptide, wherein the linker sequence does not encode for the coiled-coil domain and/or the B-box domain of a TRIM polypeptide. For example, the linker sequence provided between the RING domain and protein targeting domain may comprise the sequence GTQGERGLKKMLRTC (SEQ ID NO: 40). In one embodiment the sequence consists of the sequence GTQGERGLKKMLRTC (SEQ ID NO: 40).

Therefore one embodiment of the invention comprises a fusion protein comprising a first RING domain; a second RING domain; a PRYSPRY domain located at the C-terminal end of the first and second RING domains, and a linker sequence between the RING domains and the PRYSPRY domain comprising the sequence GTQGERGLKKMLRTC (SEQ ID NO: 40), wherein the RING domains and PRYSPRY domains are derived from a TRIM polypeptide, preferably TRIM21 and preferably wherein the fusion protein does not comprise a coiled-coil domain or a B-box domain. Preferably the RING domains comprise at least amino acids 1-81, more preferably amino acids 1-85 of SEQ ID NO: 2 or a functional variant thereof. Preferably the PRYSPRY domain comprises the sequence of SEQ ID NO: 3 or a functional variant thereof.

A “fusion protein” and a “fusion polypeptide” refer to a polypeptide having two or more portions covalently linked together, where each of the portions is a polypeptide having a specific property, which may be the same or different. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other.

The term “fusion protein” in this text means, in general terms, one or more proteins joined together by chemical means, including hydrogen bonds or salt bridges, or by peptide bonds through protein synthesis or both. Typically, fusion proteins will be prepared by DNA recombination techniques standard in the art and may be referred to herein as recombinant fusion proteins.

The invention also provides nucleic acid constructs encoding a fusion protein of the invention. The nucleic acid construct can comprise a first nucleic acid sequence encoding a first RING domain; a second nucleic acid sequence encoding a second RING domain; and a third nucleic acid sequence encoding a protein targeting domain.

Preferably the nucleic acid construct encodes for a fusion protein wherein the protein targeting domain is located at the C-terminal end of the first and second RING domains. Preferably the nucleic acid does not comprise a sequence encoding a B-box domain and/or a coiled-coil domain. The nucleic acid construct can also encode for any linker sequences located between the RING domains and/or the RING domains and protein targeting domain.

In one embodiment invention, the nucleic acid construct can comprise a first nucleic acid sequence encoding a first RING domain; a second nucleic acid sequence encoding a second RING domain; and a third nucleic acid sequence encoding a protein targeting domain wherein the nucleic acid construct does not comprise a sequence encoding for a B-box domain or a coiled-coil domain. The nucleic acid construct can encode for RING domains and protein targeting domains as described above.

In a preferred embodiment the nucleic acid construct comprises a first nucleic acid sequence encoding a first RING domain; a second nucleic acid sequence encoding a second RING domain; and a third nucleic acid sequence encoding a PRYSPRY domain, wherein the first and second RING domains are derived from TRIM, preferably TRIM21. More preferably the nucleic acid constructs do not comprise a sequence encoding for a B-box domain and/or a coiled-coil domain from TRIM, preferably derived from any polypeptide, even more preferably the fusion does not comprise a B-box domain nor a coiled-coil domain derived from TRIM, preferably derived from any polypeptide.

In one embodiment the nucleic acid construct comprises a first nucleic acid sequence encoding a first RING domain; a second nucleic acid sequence encoding a second RING domain; and a third nucleic acid sequence encoding a VHH, wherein the first and second RING domains are derived from TRIM, preferably TRIM21, and the VHH binds to a protein of interest. More preferably the nucleic acid construct does not comprise a sequence encoding for a B-box domain and/or a coiled-coil domain from TRIM, preferably derived from any polypeptide, even more preferably the fusion does not comprise a B-box domain nor a coiled-coil domain derived from TRIM, preferably derived from any polypeptide.

A nucleic acid construct can comprise a first nucleic acid sequence encoding a first RING domain; a second nucleic acid sequence encoding a second RING domain; a third nucleic acid sequence encoding a PRYSPRY domain located at the C-terminal end of the first and second RING domains, and a fourth nucleic acid sequence encoding a linker sequence comprising the sequence GTQGERGLKKMLRTC (SEQ ID NO: 40), wherein the RING domains and PRYSPRY domains are derived from a TRIM polypeptide, preferably TRIM21, and preferably wherein the nucleic acid does not comprise sequences encoding a coiled-coil domain or encoding a B-box domain. Preferably the first and second nucleic acid sequences encode for RING domains comprising at least amino acids 1-81, more preferably amino acids 1-85 of SEQ ID NO: 2 or a functional variant thereof. Preferably the third nucleic acid sequence encodes for a PRYSPRY domain comprising the sequence of SEQ ID NO: 3 or a functional variant thereof.

The invention also provides fusion proteins encoded by these nucleic acid constructs.

There nucleic acid construct may be provided in the form of a vector, for example, an expression vector, and may include, among others, chromosomal, episomal and virus-derived vectors, for example, vectors derived from bacterial plasmids, from bacteriophage, from transposons, from yeast episomes, from insertion elements, from yeast chromosomal elements, from viruses such as baculo-viruses, papova-viruses, such as SV40, vaccinia viruses, adenoviruses, lentiviruses, fowl pox viruses, pseudorabies viruses and retroviruses, and vectors derived from combinations thereof, such as those derived from plasmid and bacteriophage genetic elements, such as cosmids and phagemids. Generally, any vector suitable to maintain, propagate or express nucleic acid to express a polypeptide in a host, may be used for expression in this regard. The vector may comprise a plurality of the nucleic acid constructs defined above, for example two or more. Preferably the vector is viral delivery vector, preferably an adenoassociated virus (AAV) vector or a lentivirus vector.

The nucleic acid construct of the invention preferably includes a promoter or other regulatory sequence which controls expression of the nucleic acid. The promoter or other regulatory sequences can be operably linked to the nucleic acid sequences encoding the domains of the fusion protein. Promoters and other regulatory sequences which control expression of a nucleic acid have been identified and are known in the art. The person skilled in the art will note that it may not be necessary to utilise the whole promoter or other regulatory sequence. Only the minimum essential regulatory element may be required and, in fact, such elements can be used to construct chimeric sequences or other promoters.

The term “nucleic acid construct” generally refers to any length of nucleic acid which may be DNA, cDNA or RNA such as mRNA obtained by cloning or produced by chemical synthesis. The DNA may be single or double stranded. Single stranded DNA may be the coding sense strand, or it may be the non-coding or anti-sense strand. For therapeutic use, the nucleic acid construct is preferably in a form capable of being expressed in the subject to be treated.

The invention also provides hosts cell comprising such nucleic acid constructs.

The invention also provides a method for preparing fusion proteins of the invention, the method comprising cultivating or maintaining a host cell comprising the nucleic construct or vector described above under conditions such that said host cell produces the fusion protein, optionally further comprising isolating the fusion protein.

Also provided is a pharmaceutical composition comprising the fusion protein or nucleic acid constructs of the invention. The pharmaceutical composition may contain a variety of pharmaceutically acceptable carriers and/or excipients. Suitable pharmaceutically acceptable carriers and/or excipients are known in the art. Pharmaceutical compositions of the invention may be for administration by any suitable method known in the art, including but not limited to intravenous, intramuscular, oral, intraperitoneal, or topical administration. In preferred embodiments, the pharmaceutical composition may be prepared in the form of a liquid, gel, powder, tablet, capsule, or foam.

The fusion proteins and nucleic acid constructs of the invention may be used for therapy as a medicament. In one embodiment the invention also provides for the treatment of neurological disorders, for example Alzheimer's Disease or Huntington's Disease. In other embodiments the invention provides for the treatment of an infection, for example a viral infection such as HIV. In further embodiments the invention provides for the treatment of a trinucleotide repeat disorder, in particular trinucleotide repeat disorders wherein the trinucleotide repeat resides in the coding sequence of the gene. Trinucleotide repeat disorders that may be treated with the fusion proteins or nucleic acid constructs of the invention include Huntington disease, Dentatorubropallidoluysian atrophy and spinocerebellar ataxia.

The treatment of the neurological disorder, infection or trinucleotide repeat disorder comprises administering to the subject a fusion protein, nucleic acid or pharmaceutical composition of the invention.

In one embodiment the treatment involves administering a fusion protein comprising: a first RING domain; a second RING domain; and a protein targeting domain. Preferably the protein targeting domain is located at the C-terminal end of the first and second RING domains. Preferably the fusion protein administered does not comprise a coiled-coil domain or a B-box domain.

In one embodiment of the invention the treatment involves administering a nucleic acid construct comprising a first nucleic acid sequence encoding a first RING domain; a second nucleic acid sequence encoding a second RING domain; and a third nucleic acid sequence encoding a protein targeting domain. Preferably the nucleic acid construct encodes for a fusion protein wherein the protein targeting domain is located at the C-terminal end of the first and second RING domains. Preferably the nucleic acid constructs administered do not comprise a sequence encoding for a B-box domain or a coiled-coil domain.

When the disorder to be treated is a neurological disorder such as Alzheimer Disease, the protein targeting domain may encode for a sequence that targets tau. In one embodiment the protein targeting domain may encode for an antibody, antibody fragment thereof or antibody mimetic that specifically binds for tau. When the disorder to be treated is Huntington disease, the protein targeting domain may encode for a sequence that targets huntingtin. In one embodiment the protein targeting domain may encode for an antibody, antibody fragment thereof or antibody mimetic that specifically bind the polyglutamate sequence of huntingtin.

The nucleic acid construct according to the invention may also be administered by means of delivery vectors. These include viral delivery vectors, such as adenovirus, retrovirus or lentivirus delivery vectors known in the art. Other non-viral delivery vectors include lipid delivery vectors, including liposome delivery vectors known in the art.

Treatment includes both prophylaxis (prevention) and therapeutic treatment. The terms “treat”, “treating” or “treatment” (or equivalent terms) mean that the severity of the individual's condition is reduced or at least partially improved or ameliorated and/or that some alleviation, mitigation or decrease in at least one clinical symptom is achieved and/or there is an inhibition or delay in the progression of the condition and/or prevention or delay at the onset of a disease or illness.

The terms “patient”, “individual” or “subject” include human and other mammalian subjects that receive either prophylactic or therapeutic treatment with the fusion proteins or nucleic acid constructs described herein. Mammalian subjects include primates, e.g., non-human primates. Mammalian subjects also include laboratory animals commonly used in research, such as but not limited to, rabbits and rodents such as rats and mice.

The fusion proteins and nucleic acid constructs of the invention may also be used as a research tool, for example the degradation of proteins in a cell or sample.

Accordingly, in one embodiment of the invention there is provided a method of degrading a protein in a cell comprising administering a fusion protein or a nucleic acid of the invention. The cell may be an in vitro cell.

A further embodiment of the invention provides a method of degrading a protein in a sample comprising introducing a fusion protein or a nucleic construct of the invention into a sample.

In one embodiment the methods of degrading a protein in a cell or sample involves administering a fusion protein comprising: a first RING domain; a second RING domain; and a protein targeting domain. Preferably the protein targeting domain is located at the C-terminal end of the first and second RING Domain. Preferably the fusion protein administered does not comprise a coiled-coil domain or a B-box domain.

In one embodiment of the invention the methods of degrading a protein in a cell or sample involve administering a nucleic acid construct comprising a first nucleic acid sequence encoding a first RING domain; a second nucleic acid sequence encoding a second RING domain; and a third nucleic acid sequence encoding a protein targeting domain. Preferably the nucleic acid constructs administered does not comprise a sequence encoding for a B-box domain or a Coiled-coil domain.

An antibody, antibody fragment thereof, or antibody mimetic targeting a protein of interest, or a nucleic acid encoding the antibody, antibody fragment thereof, or antibody mimetic, may also be administered to the cell or sample. A “protein of interest” is a protein targeted for degradation. The antibody, antibody fragment thereof, or antibody mimetic may specifically bind the protein of interest.

The methods are particular useful for degrading proteins in cells that don't endogenously express TRIM21. The methods of are particularly useful in degrading intracellular proteins. However, in some embodiments an antibody will bind the protein of interest extracellularly, for example when targeting a pathogen, such as a virus. The antibody-target will be internalised in a cell, where the fusion protein will bind the antibody-target degrading the protein.

The fusion protein or nucleic acid can be introduced into the cell by transfection for example by injection, including microinjection or by electroporation, or transduction for example by the use of a viral delivery vector, for example an AAV vector. Other suitable delivery techniques for introducing the fusion protein and nucleic acid constructs into cells are known in the art.

The phrase “selected from the group comprising” may be substituted with the phrase “selected from the group consisting of” and vice versa, wherever they occur herein.

The contents of all publications cited herein are incorporated herein by reference in their entirety into this application to more fully describe the state of the art to which this invention pertains.

The present invention will be further understood by reference to the following examples.

### EXAMPLES

### Example 1: Structure of Mono-ubiquitinated TRIM21 RING Domain in Complex With E2 Heterodimer Ube2N/Ube2V2

### Materials & Methods

Plasmids: Bacterial expression constructs: Ube2V2 and TRIM21 expression constructs but full-length were cloned into pOP-TG vectors and full-length TRIM21 constructs into HLTV vectors. Ube2N constructs were cloned into pOP-TS, Ube1 into pET21 and ubiquitin into pET17b. Ube2D1 was cloned into pET28a. For cloning Ub_{4/3/2}-TRIM21 constructs, a linear Ub₃ sequence was codon optimized, ordered as synthetic DNA (Integrated DNA technologies, Coralville, Iowa, USA) and inserted into the Ub^{G75/76A}-TRIM21 construct. All constructs for mRNA production were cloned into pGEMHE vectors. Constructs were cloned by Gibson Assembly and mutations were inserted by mutagenesis PCR. For mCherry-TRIM21^{ΔRING-Box}, TRIM21³⁸²⁻¹⁴²⁸ was amplified by PCR and cut by EcoRI and Notl. A 743 bp fragment carrying mCherry was cut by Agel and EcoRI from V60 (pmCherry-C1, Clonetech) and both fragments were ligated into pGEMHE. The sequences of the purified protein/expressed mRNA are provided in SEQ ID Nos: 6-38.

Expression and purification of recombinant proteins: Ubiquitin-TRIM21, TRIM21 RING (residue 1-85), Ube2N and Ube2V2 constructs were expressed in Escherichia coli BL21 DE3 cells. Ubiquitin and Ube1 were expressed in E. coli Rosetta 2 DE3 cells. All cells were grown in 2xTY media supplemented with 2 mM MgSO₄, 0.5% glucose and 100 μg mL⁻¹ ampicillin (and 35 μg mL⁻¹ chloramphenicol for expression is Rosetta 2 cells). Cells were induced at an OD⁶⁰⁰ of 0.7. For TRIM proteins, induction was performed with 0.5 mM IPTG and 10 μM ZnCl₂, for ubiquitin and Ube1 with 0.2 mM IPTG and for E2 enzymes with 0.5 mM IPTG. After centrifugation, cells were resuspended in 50 mM Tris pH 8.0, 150 mM NaCl, 10 μM ZnCl₂, 1 mM DTT, 20% Bugbuster (Novagen) and c0mplete™ protease inhibitors (Roche, Switzerland). Lysis was performed by sonication. TRIM proteins and Ube2V2 were expressed with N-terminal GST-tag and purified via glutathione sepharose resin (GE Healthcare) equilibrated in 50 mM Tris pH 8.0, 150 mM NaCl and 1 mM DTT. The tag was cleaved on beads overnight at 4° C. In case of Ubiquitin-TRIM21 constructs, the eluate was supplemented with 10 mM imidazole and run over 0.25 mL of Ni-NTA beads to remove His-tagged TEV. Ube2N and Ube1 were expressed with an N-terminal His-tag and were purified via Ni-NTA resin. Proteins were eluted in 50 mM Tris pH 8.0, 150 mM NaCl, 1 mM DTT and 300 mM imidazole. For Ube2N, TEV-cleavage of the His-tag was performed over-night by dialyzing the sample against 50 mM Tris pH 8.0, 150 mM NaCl, 1 mM DTT and 20 mM imidazole. Afterwards, His-tagged TEV protease was removed by Ni-NTA resin. The cleavage left an N-terminal tripeptide scar (GSH) on recombinantly expressed TRIM proteins, an N-terminal G scar on Ube2N and an N-terminal GSQEF scar on Ube2V2. Finally, size exclusion chromatography was carried out on a HiLoad 26/60 Superdex 75 prep grade column (GE Healthcare) in 20 mM Tris pH 8.0, 150 mM NaCl and 1 mM DTT.

Full-length TRIM21 (Ub-R-B-CC-PS or Ub-R-R-B-CC-PS) were expressed as His-Lipoyl-fusion proteins in E. coli BL21 DE3 cells. Cells in 2xTY were grown to an OD600 of 0.8 and induced with 0.5 mM IPTG and 10 μM ZnCl₂. Cells were further incubated at 18° C., 220 rpm overnight. After centrifugation, cells were resuspended in 100 mM Tris pH 8.0, 250 mM NaCl, 10 μM ZnCl₂, 1 mM DTT, 20% Bugbuster (Novagen), 20 mM Imidazole and c0mplete™ protease inhibitors (Roche, Switzerland). Lysis was performed by sonication. His-affinity purification was performed as described above. Immediately afterwards, the protein was applied to an S200 26/60 column (equilibrated in 50 mM Tris pH 8.0, 200 mM NaCl, 1 mM DTT) to remove soluble aggregates. After concentration determination, the His-Lipyol tag was cleaved using TEV protease overnight. Since full-length TRIM21 is unstable without tag, the protein was not further purified but used for assays.

Ubiquitin purification was performed following the protocol established by the Pickart lab (Pickart, C. M. & Raasi, S. Controlled synthesis of polyubiquitin chains. Methods Enzymol 399, 21-36, (2005). After cell lysis by sonication (lysis buffer: 50 mM Tris pH 7.4, 1 mg mL⁻¹ Lysozyme (by Sigma Aldrich, St. Louis, USA), 0.1 mg mL⁻¹ DNAse (by Sigma Aldrich, St. Louis, USA)), a total concentration of 0.5% percloric adic was added to the stirring lysate at 4° C. The (milky) lysate was incubated for another 30 min on a stirrer at 4° C. to complete precipitation. Next, the lysate was centrifuged (50,000 xg) for 30 min at 4° C. The supernatant was dialyzed overnight (3500 MWCO) against 3 L 50 mM sodium acetate (NaOAc) pH 4.5. Afterwards, Ub was purified via cation-exchange chromatography using a 20 mL SP column (GE Healthcare) using a NaCl gradient (0-1000 mM NaCl in 50 mM NaOAc pH 4.5). Finally, size exclusion chromatography was carried out on a HiLoad 26/60 Superdex 75 prep grade column (GE Healthcare) in 20 mM Tris pH 7.4.

All proteins were flash frozen in small aliquots (30-100 μL) and stored at −80° C.

Formation of an isopeptide-linked Ube2N˜Ub: Ube2N^{C87K/K92A} charging with WT ubiquitin was performed as normal E1-mediated charging but in a high pH to ensure K87 deprotonation. The isopeptide charging reaction was carried out in 50 mM Tris pH 10.0, 150 mM NaCl, 5 mM MgCl₂, 0.5 mM TCEP, 3 mM ATP, 0.8 μM Ube1, 100 μM Ube2N and 130 μM ubiquitin at 37° C. for 4 hours. After conjugation, Ube2N^{C87K/K92A}˜Ub was purified by size exclusion chromatography (Superdex S75 26/60, GE Healthcare) that was equilibrated in 20 mM Tris pH 8.0 and 150 mM NaCl.

Crystallization: In total, 5 mg mL⁻¹ of human Ub^{G75/76A}-TRIM21¹⁻⁸⁵, Ube2N^{C87K/K92A}˜Ub and Ube2V2 in 20 mM Tris pH 8.0, 150 mM NaCl and 1 mM DTT were subjected to sparse matric screening in sitting drops at 17° C. (500 nL protein was mixed with 500 nL reservoir solution). Crystals were obtained in Morpheus II screen (Gorrec, F. The MORPHEUS II protein crystallization screen. Acta Crystallogr F Struct Biol Commun 71, 831-837, (2015)) in 0.1 M MOPSO/bis-tris pH 6.5, 12.5% (w/v) PEG 4K, 20% (v/v) 1,2,6-hexanetriol, 0.03 M of each Li, Na and K.

For the Ube2N^{C87K/K92A}˜Ub:Ube2V2 structure, 10 mg mL⁻¹ TRIM21¹⁻⁸⁵, Ube2N^{C87K/K92A}˜Ub, Ube2V2 and Ub in 20 mM Tris pH 8.0, 150 mM NaCl and 1 mM DTT were subjected to sparse matrix screening in sitting drops at 17° C. (200 nL protein was mixed with 200 nL reservoir solution). Crystals were obtained in the Morpheus III screen (Sammak, S. et al. Crystal Structures and Nuclear Magnetic Resonance Studies of the Apo Form of the c-MYC:MAX bHLHZip Complex Reveal a Helical Basic Region in the Absence of DNA. Biochemistry 58, 3144-3154, (2019)) in 0.1 M bicine/Trizma base pH 8.5, 12.5% w/v PEG 1000, 12.5% w/v PEG 3350, 12.5% v/v MPD, 0.2% (w/v) of each Anesthetic alkaloids (lidocaine HCl·H₂O, procaine HCl, proparacaine HCl, tetracaine HCl). Crystals were flash frozen for data collection without the use of additional cryo-protectant.

Crystal data collection, structure solution and refinement: Data were collected at the Diamond Light Source beamline i03, equipped with an Eiger2 XE 16M detecter of a wavelength of 0.9762 Å. For Ub^{G75/76A}-TRIM21¹⁻⁸⁵:Ube2N^{C87K/K92A}˜Ub:Ube2V2, Diffraction images were processed using XDS (Kabsch, W. Xds. Acta Crystallogr D Biol Crystallogr 66, 125-132, (2010)) to 2.2 Å resolution. The crystals belong to space group number 5 (C2) with each of the components present as a single copy in the asymmetric unit. Analysis of the raw data revealed moderate anisotropy in the data. The structure was solved by molecular replacement using PHASER-MR implemented in the Phenix suite (Adams, P. D. et al. PHENIX: a comprehensive Python-based system for macromolecular structure solution. Acta Crystallogr D Biol Crystallogr 66, 213-221, (2010)). Search models served TRIM21-RING and Ube2N from 6S53 (Kiss, L. et al. A tri-ionic anchor mechanism drives Ube2N-specific recruitment and K63-chain ubiquitination in TRIM ligases. Nat Commun 10, 4502, (2019)), ubiquitin from 1UBQ (Vijay-Kumar, S., Bugg, C. E. & Cook, W. J. Structure of ubiquitin refined at 1.8 A resolution. J Mol Biol 194, 531-544, (1987)) and Ube2V2 from 1J74 (Moraes, T. F. et al. Crystal structure of the human ubiquitin conjugating enzyme complex, hMms2-hUbc13. Nat Struct Biol 8, 669-673, (2001)). Model building and real-space-refinement was carried out in coot (Emsley, P. & Cowtan, K. Coot: model-building tools for molecular graphics. Acta Crystallogr D Biol Crystallogr 60, 2126-2132, (2004)), and refinement was performed using phenix-refine (Afonine, P. V. et al. Towards automated crystallographic structure refinement with phenix.refine. Acta Crystallogr D Biol Crystallogr 68, 352-367, (2012)). The anisotropy in the data could be observed in parts of the map that were less well resolved. While all interfaces show clear high-resolution density, particularly parts of Ube2V2 (chain A) that were next to a solvent channel proved challenging to build. The structure is deposited in the Protein Data Bank under the accession code 7BBD [http://doi.org/10.2210/pdb7BBD/pdb].

For Ube2N^{C87K/K92A}˜Ub:Ube2V2, diffraction images were processed using XDS to 2.54 Å resolution. The crystals belong to space group number 145 (P3₂) with each component present three times in the asymmetric unit, related by translational non-crystallographic symmetry. The structure was solved by PHASER-MR implemented in the Phenix suite. Search models used were Ube2N from 6S53, ubiquitin from 1UBQ and Ube2V2 from 1J74. Model building and real-space-refinement was carried out in coot, and refinement was performed using phenix-refine. The structure is deposited in the Protein Data Bank under the accession code 7BBF [http://doi.org/10.2210/pdb7BBF/pdb].

### Results

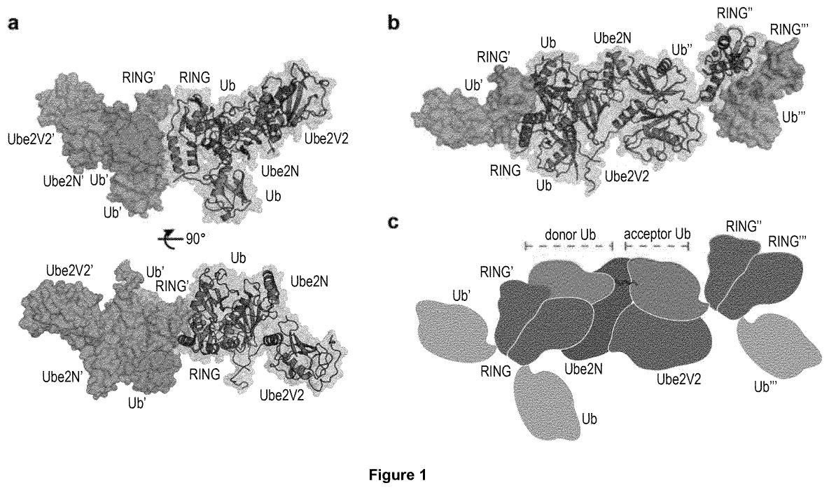

We set out to understand how a substrate-bound ubiquitin chain can be formed. In principle, ubiquitin chain elongation of TRIM proteins depends on their RING domain only. In the case of TRIM21 (and TRIM5α), the TRIM RING itself is the substrate, after it has undergone N-terminal mono-ubiquitination upon interaction with the E2 enzyme Ube2W. Therefore, we attempted to address substrate-bound ubiquitination with TRIM21 RING and its chain forming E2 heterodimer Ube2N/Ube2V2. In crystallization trials, we used N-terminally mono-ubiquitinated TRIM21 RING domain (Ub^{G75/76A}-TRIM21¹⁻⁸⁵ or Ub-R), an isopeptide-linked, non-hydrolyzable ubiquitin-charged Ube2N conjugate (Ube2N˜Ub) and Ube2V2. We solved the atomic structure of this complex at 2.2 Å resolution, with one copy each of Ub-R, Ube2N˜Ub and Ube2V2 in the asymmetric unit (data not shown). The naturally occurring TRIM21 RING homo-dimer was generated in our model by invoking crystal symmetry (FIG. 1a). The RINGs engage Ube2N˜Ub in the closed conformation and Ube2N forms a heterodimer with Ube2V2. Analyzing further interactions within the crystal lattice, we found that the TRIM21-linked ubiquitin made additional contacts to Ube2N/Ube2V2 of a symmetry related complex (FIG. 1b), which orient the RING-bound ubiquitin so that its K63 points towards the active site, ready for nucleophilic attack (FIG. 1b, c). Our structure thus represents a snapshot of a ubiquitin-primed RING ready for self-anchored ubiquitin chain elongation.

### Example 2: Chemical Mechanism of Ubiquitination

### Materials & Methods

Kinetics of di-ubiquitin formation: Purified proteins were obtained as described above. Kinetic measurements of di-ubiquitin formation were measured for Michaelis-Menten, and pKₐ analysis. The experiment was performed in a pulse-chase format, where the first reaction generated Ube2N˜^{His-}Ub and was chased by Ub¹⁻⁷⁴. Under these conditions, Ub¹⁻⁷⁴ only acts as acceptor, as it cannot be charged onto the E1 enzyme. His-tagged ubiquitin on the other hand serves as donor. Although, theoretically His-Ub could also act as an acceptor, the high concentrations of Ub¹⁻⁷⁴ outcompete His-Ub as an acceptor. Initially, we determined the linear range of the reaction for all different constructs, so as to later measure only one point on this trajectory as a representative for the initial velocity (v₀). For Michaelis-Menten kinetics we used the following length: WT, 3 min; D119A, 100 min; D119N, 30 min; N123A, 3 min; D124A. 3 min, and for pKₐ measurements the following: WT, 40 s; D119A, 5 min; D119N, 60 s; N123A, 40 s; D124A, 40 s.

First, Ube2N-charging was performed in 50 mM Tris pH 7.0, 150 mM NaCl, 20 mM MgCl₂, 3 mM ATP, 60 μM His-ubiquitin, 1 μM GST-Ube1 (Boston Biochem) and 40 μM Ube2N. The reaction was incubated at 37° C. for 12 min and stored afterwards at 4° C. until use (within 1 h).

For Michaelis-Menten kinetic analysis, the reaction was conducted in 50 mM Tris pH 7.4, 150 mM NaCl with the indicated amount of Ub¹⁻⁷⁴ (0-400 μM), while for pKₐ determination in 50 mM Tris and the indicated pH (7.0-10.5), 50 mM NaCl and 250 mM Ub¹⁻⁷⁴. Apart from the buffer, the reaction mix contained 2.5 μM Ube2V2. The reaction was initiated by addition of charging mix that was diluted 1 in 20, resulting in 2 μM Ube2N in the reaction. The reaction was stopped by addition of 4×LDS loading buffer. The samples were boiled at 90° C. for 2 min and resolved by LDS-PAGE. Western blot was performed with anti-His antibody (Clontech, 631212, 1:5000) via the LiCor system, leading to detection of the following species: His-Ub, His-Ub-Ub¹⁻⁷⁴, Ube2N˜^{His-}Ub, Ube2N˜(^{His-}Ub)₂ (a side product of the charging reaction that shows ubiquitination rates similar to Ube2N˜^{His-}Ub) and E1-^{His-}Ub. The concentration of ^{His-}Ub-Ub¹⁻⁷⁴ was determined by dividing the value for ^{His-}Ub-Ub¹⁻⁷⁴ by the sum of all bands detected and multiplying this by the total concentration of ^{His-}Ub in the reaction (3 μM). Experiments were performed in technical triplicates. Michaelis-Menten kinetics data were fit to Equation (1):

$$V = \frac{{E}_{t} * {k}_{cat} * S}{{K}_{M} * S} \qquad ( 1 )$$

where V is the measured velocity, Eₜ the total concentration of active sites (2 μM) and S the substrate concentration. The curve was fit to determine k_{cat} and K_{M}. To determine the pKₐ, the data was fit to Equation (2):

$$V = \frac{{V}_{HA} * {10}^{- p H} + {V}_{A -} * {10}^{- {pK}_{a}}}{1 {0}^{- {pK}_{a}} + {10}^{- p H}} \qquad ( 2 )$$

as given in⁶⁵, where V is the measured velocity, V_{A-} the velocity for the basic species and V_{HA} the velocity for the acidic species.

### Results

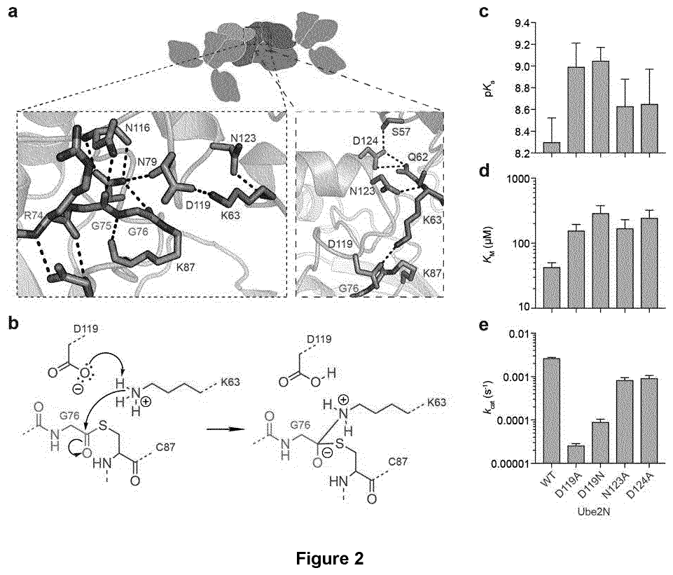

Having captured a 2.2 Å resolution representation of the system prior to catalysis, we were able to perform a detailed analysis of ubiquitin transfer. The Ube2N-charged ubiquitin can be found in the RING-promoted closed Ube2N˜Ub conformation and thus represents the donor ubiquitin (FIG. 1). The RING-bound ubiquitin of a symmetry related complex was captured by Ube2N/Ube2V2, positioning its nucleophilic K63 N^{ζ}H₃group 4.8 Å from the electrophilic carbonyl of the donor ubiquitin C-terminus (FIG. 2a). Interestingly, K63 of this acceptor ubiquitin shows a direct interaction with D119 of Ube2N (FIG. 2a). This suggests that D119 deprotonates K63 on the acceptor ubiquitin, thereby activating it for nucleophilic attack. Indeed, the corresponding residue in Ube2D (D117) has been suggested to be involved in positioning and/or activating an incoming acceptor lysine (Plechanovova, A., Jaffray, E. G., Tatham, M. H., Naismith, J. H. & Hay, R. T. Structure of a RING E3 ligase and ubiquitin-loaded E2 primed for catalysis. Nature 489, 115-120, (2012)).

To investigate the chemical mechanism of ubiquitination (FIG. 2b), we measured the kinetics of di-ubiquitin formation. The acid coefficient (pKₐ) of this reaction should solely depend on the protonation state of its nucleophile, K63. Fitting the ubiquitination velocity of reactions carried out at different pHs to an equation assuming one titratable group revealed a pKₐ of 8.3 for Ube2N (FIG. 2c), comparable to what was observed for the SUMO-E2 Ube2I (Yunus, A. A. & Lima, C. D. Lysine activation and functional analysis of E2-mediated conjugation in the SUMO pathway. Nat Struct Mol Biol 13, 491-499, (2006)). This is significantly lower than the pKₐ of 10.5 for a free lysine ζ-amino group (Lide, D. R. CRC handbook of chemistry and physics: a ready-reference book of chemical and phyical data. Vol. 72 (CRC Press, 1991)), which would be incompatible with catalysis at physiological pH˜7.34 (Llopis, J., McCaffery, J. M., Miyawaki, A., Farquhar, M. G. & Tsien, R. Y. Measurement of cytosolic, mitochondrial, and Golgi pH in single living cells with green fluorescent proteins. Proc Natl Acad Sci U S A 95, 6803-6808 (1998)). We mutated D119 to either alanine or asparagine as neither can act as a base, but asparagine could still bind and orient K63. Both mutants increased the pKₐ to ˜9 (FIG. 2c). At physiological pH, Ube2N^{D119A/N} modestly increased the K_{M} by ˜4 and ˜7-fold, respectively (FIG. 2d). Mutation to alanine reduced k_{cat} 100-fold and mutation to asparagine 30-fold (FIG. 2e), suggesting that substrate turnover also depends on orientation of the lysine nucleophile. Yet, this catalytic rate does not yield efficient ubiquitin chain formation under physiological pH (data not shown). Together, these observations establish that D119 is the base that deprotonates the incoming acceptor lysine to enable catalysis.

Interactions between ubiquitin and other proteins have been shown to depend on specific conformations of ubiquitin's β₁-β₂ loop, which can be found in either loop-in or loop-out conformations (Hospenthal, M. K., Freund, S. M. & Komander, D. Assembly, analysis and architecture of atypical ubiquitin chains. Nat Struct Mol Biol 20, 555-565, (2013)). These motions change the ubiquitin core structure and subsequent conformational selection enables ubiquitin to interact with many different binding partners (Lange, O. F. et al. Recognition dynamics up to microseconds revealed from an RDC-derived ubiquitin ensemble in solution. Science 320, 1471-1475, (2008)). In our structure, we found the donor ubiquitin β₁-β₂ loop in its loop-in configuration, and loop-out to be incompatible with formation of the closed conformation (data not shown). Conversely, the acceptor ubiquitin was in a loop-out configuration (data not shown), which appears to be the default state in ubiquitin (Hospenthal et al. 2013). Donor and acceptor ubiquitin also have distinct B-factor profiles (data not shown), perhaps reflecting some other aspect of their different roles in catalysis. Interestingly, the β₁-β₂ loop conformation also appears to be critical in ubiquitin-like proteins such as Nedd8, when activating cullin-RING-ligases (CRL) (Baek, K. et al. NEDD8 nucleates a multivalent cullin-RING-UBE2D ubiquitin ligation assembly. Nature 578, 461-466, (2020)).

RING E3s act by locking the normally very dynamic E2˜Ub species in a closed conformation, thereby priming it for catalysis (FIG. 2a). Comparison with our previously determined TRIM21 R:Ube2N˜Ub structure (Kiss, L. et al. A tri-ionic anchor mechanism drives Ube2N-specific recruitment and K63-chain ubiquitination in TRIM ligases. Nat Commun 10, 4502, (2019)) shows scarcely any difference between the donor ubiquitin C-termini and the Ube2N active site (data not shown). Nonetheless, formation of the closed Ube2N˜Ub conformation alone is not sufficient for catalysis, as this also requires the presence of Ube2V2, which binds and orients the acceptor ubiquitin. We gained additional insight into how Ube2V2 positions the acceptor ubiquitin by analysing a Ube2N˜Ub:Ube2V2 complex that we solved at 2.5 Å resolution (data not shown). By invoking crystal symmetry, this structure shows the orientation of an acceptor ubiquitin by Ube2V2, so that its K63 is pointed towards the active site of Ube2N (data not shown), an orientation comparable with a structure of yeast Ube2N˜Ub:Ube2V2 that was solved in a different crystal lattice (Eddins, M. J., Carlile, C. M., Gomez, K. M., Pickart, C. M. & Wolberger, C. Mms2-Ubc13 covalently bound to ubiquitin reveals the structural basis of linkage-specific polyubiquitin chain formation. Nat Struct Mol Biol 13, 915-920, (2006)). Without a RING present, the donor ubiquitin is not in the closed conformation and our Ube2N˜Ub:Ube2V2 structure thus represents an inactive complex. Alignment to our Ub-R:Ube2N˜Ub:Ube2V2 structure (data not shown) reveals that Ube2N and Ube2V2 are packed more closely against each other, resulting in additional contacts between the acceptor ubiquitin and Ube2N (FIG. 2a) that position the nucleophile K63 much nearer to the active site (4.8 Å vs. 7.5 Å). This is achieved because Ube2N N123 and D124 contact ubiquitin via the amide of ubiquitin K63 and the sidechains of S57 and Q62, respectively (FIG. 2a). The ˜3-fold reduction in k_{cat} (FIG. 2e) for the mutants Ube2N^{N123A} and Ube2N^{D124A} suggest that the function of these residues is to finetune the ubiquitination reaction by aiding orientation of the nucleophile. Taken together, the features of our structure trapped in the process of ubiquitin chain formation provide mechanistic insight into how the RING E3 promotes catalysis by simultaneously activating Ube2N for ubiquitin discharge and allowing Ube2V2 to precisely orient the acceptor ubiquitin.

### Example 3: Mechanism of RING-anchored Ubiquitination

### Materials & Methods

Ubiquitin chain formation assay: Ubiquitin chain formation assays were performed in 50 mM Tris pH 7.4, 150 mM NaCl, 2.5 mM MgCl₂ and 0.5 mM DTT. The reaction components were 2 mM ATP, 0.25 μM Ube1, 80 μM ubiquitin, 0.5 μM Ube2N/Ube2V2 or Ube2D1 together with the indicated concentration of E3. Samples were taken at the time points indicated and the reaction was stopped by addition of LDS sample buffer at 4° C. The samples were boiled at 90° C. for 2 min and resolved by LDS-PAGE. Ubiquitin chains were detected in the western blot using an anti-Ub-HRP (Santa Cruz, sc8017-HRP P4D1, 1:5,000), TRIM21 by rabbit anti-TRIM21^{PRYSPRY} D101D ST #9204 (1:1,000) and Fc by goat anti-human IgG-Fc broad 5211-8004 (1:2,000).

### Results

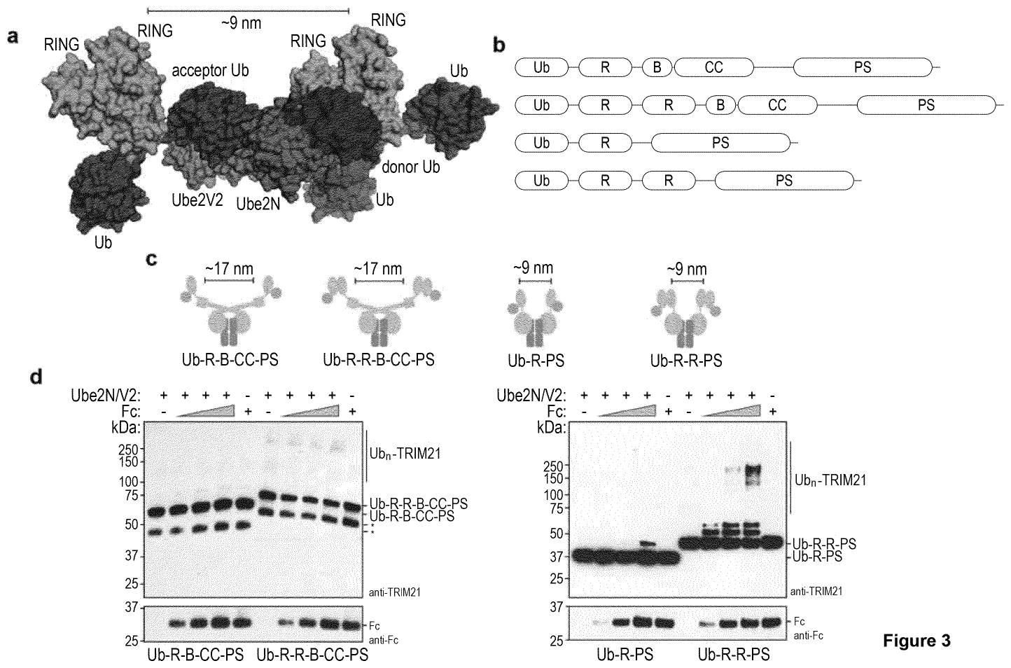

Next, we sought to understand how RING-anchored ubiquitin chains are formed. In our crystal structure, one RING dimer is positioned so as to mediate the elongation of another mono-ubiquitinated RING in trans (FIGS. 1b, 1c, 3a). Importantly, this mechanism depends only on binding of the RING-anchored acceptor ubiquitin to Ube2N/Ube2V2, as no contacts with the RING itself could be observed in our crystal structure. The relative topology of the different RING domains (enzyme and substrate) is thus mostly dictated by the catalytic interfaces, resulting in a ˜9 nm separation between the enzyme and substrate RINGs (FIG. 3a). We refer to this arrangement as the catalytic RING topology, in which a RING dimer acts as an enzyme and at least one further RING acts as the substrate for ubiquitination. This topology is not rigid since the linkers between the acceptor ubiquitin and the RING (˜3 nm apart) and the RING and the next (B-box) domain in the TRIM ligase (˜3.5 nm apart) likely provide additional flexibility (FIG. 3a, b). In our structure it is clear that initiation of TRIM21-anchored chain elongation cannot occur in cis, as the priming ubiquitin cannot reach the Ube2N/Ube2V2 binding surface (FIG. 3a). Consistent with this, we found that TRIM21 ubiquitin transfer in trans can occur in principle (data not shown), in line with previous work on TRIM5 (Fletcher, A. J. et al. Trivalent RING Assembly on Retroviral Capsids Activates TRIM5 Ubiquitination and Innate Immune Signaling. Cell Host Microbe 24, 761-775 e766, (2018)).

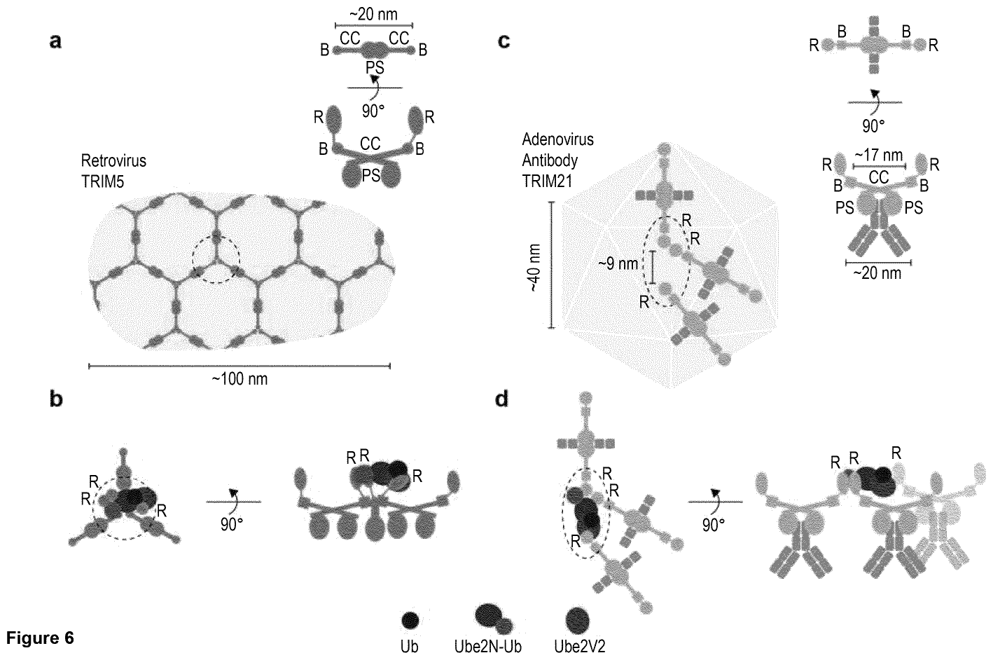

To investigate the spatial requirements of TRIM21 RING domains for self-anchored ubiquitination experimentally, we established a substrate-dependent ubiquitination assay. TRIM21 is recruited by Fc, which is an obligate dimer in solution and can be bound by two PRYSPRY (PS) domains (FIG. 7). To test for the catalytic RING topology, we designed a series of mono-ubiquitinated TRIM21 constructs that vary the number of RINGs available and their distance to each other when bound to Fc (FIG. 3b, FIG. 7). To suppress background activity, TRIM21 was used at low concentrations (100 or 50 nM) and the reaction was incubated for 5 min only. Full-length TRIM proteins form antiparallel homo-dimers via their coiled-coil domains, resulting in the separation of the two TRIM21 RING domains by ˜17 nm even when bound to Fc (FIG. 7). According to our model, addition of Fc alone should therefore not induce the catalytic RING topology (FIG. 3c). Indeed, addition of Fc did not stimulate ubiquitination of the full-length Ub-TRIM21 (Ub-RING-Box-coiled-coil-PRYSPRY or Ub-R-B-CC-PS, FIG. 3d). Even when adding an additional RING domain to make the full-length protein a constitutive RING dimer (Ub-R-R-B-CC-PS), formation of the catalytic RING topology is excluded (FIG. 3c) and no induction of self-ubiquitination is observed upon addition of Fc (FIG. 3d, FIG. 7). As a next step, we designed TRIM21 constructs lacking the B-box and coiled-coil (Ub-R-PS and Ub-R-R-PS). Fc is capable of recruiting two of these constructs, thereby locating their RINGs within ˜9 nm (FIG. 3c, FIG. 7), the distance required for the catalytic RING topology (FIG. 3a, c). Addition of Fc to Ub-R-PS led to weak self-ubiquitination. This low level of activity is likely because Ub-R-PS can only provide a monomeric RING as the enzyme, while a monomeric RING on the second Ub-R-PS acts as the substrate. TRIM RING dimerization is known to greatly increase ligase activity. We therefore repeated these experiments using a Ub-R-R-PS construct. We predicted that this should allow the catalytic RING topology observed in our crystal structure to form upon substrate binding, as the Fc will bring two RING dimers into close proximity (FIG. 3a, c). Indeed, addition of Fc to Ub-R-R-PS resulted in efficient formation of TRIM21-anchored ubiquitin chains (FIG. 3d). Importantly, while anchored ubiquitination occurred very efficiently, hardly any free ubiquitin chains could be observed (data not shown). Since self-ubiquitination only requires E2˜Ub to be recruited by the ligase, this explains its high efficiency relative to free ubiquitin chain formation, as the latter would require recruitment of both E2˜Ub and (poly-) ubiquitin. Indeed, Ub-R-R-PS worked efficiently in our substrate-induced ubiquitination assay even at reduced TRIM21 concentrations (data not shown). Thus, inducing formation of the catalytic RING topology by substrate binding enables robust and selective formation of self-anchored ubiquitin chains. Moreover, the catalytic RING topology is only achieved when the separate requirements of an active enzyme (a dimeric RING) and a correctly positioned substrate (a third RING) are fulfilled.

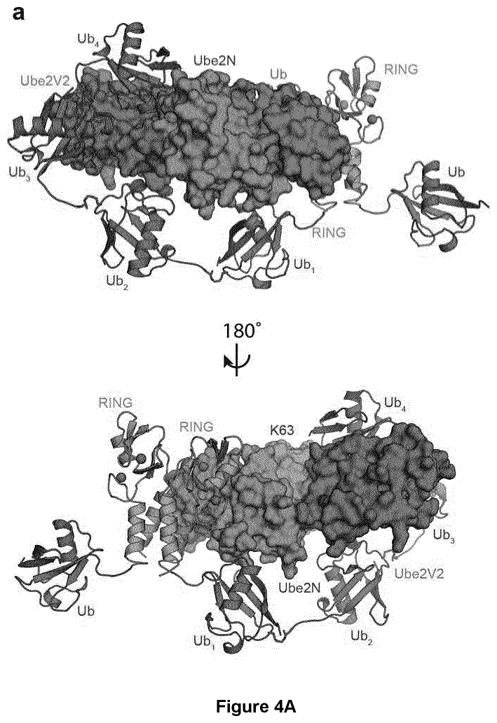

We next considered how long a TRIM21-anchored ubiquitin chain would have to be for cis ubiquitination to become sterically possible. Using our Ub-R:Ube2N˜Ub:Ube2V2 structure, we created models with increasing numbers of K63-linked ubiquitin chains conjugated to the TRIM21 RING domain. These models suggested that a chain of four ubiquitin molecules would be necessary and sufficient for self-ubiquitination in cis (FIG. 4a). Thus, after addition of the priming ubiquitin, three ubiquitin molecules must be added in trans, before the chain could be further elongated in cis. Consistent with this, we only observed very long TRIM21-anchored ubiquitin chains or species carrying one, two or three ubiquitin molecules in our Fc-dependent TRIM21 ubiquitination experiments (FIG. 3d). With the addition of a fourth ubiquitin, the reaction appears to progress much more quickly, as would be expected for a switch from trans to cis, rapidly consuming the tetra-ubiquitin species and converting it into a long chain. In the above experiments, self-ubiquitination only occurred when two Ub-R-R-PS constructs were co-localized by their binding to Fc to satisfy the requirements of the catalytic RING topology (FIG. 3c, d). To confirm the switch in self-ubiquitination from trans to cis experimentally, we generated TRIM21 R-R-PS constructs wherein their N-termini were fused to up to four linearly connected ubiquitin molecules. Due to their high structural similarity, we assumed a linear chain would mimic a K63-linked ubiquitin chain in length and flexibility sufficiently well. Upon testing these new constructs, we observed that only TRIM21 modified with tetra-ubiquitin became independent of Fc for self-ubiquitination (FIG. 4b). All the other, shorter, constructs remained rate-limited by first having to self-ubiquitinate in trans, before switching to cis. This biochemical data is in agreement with our structure showing the initiation of RING-anchored ubiquitination in trans and our model of polyubiquitinated RING elongation in cis.

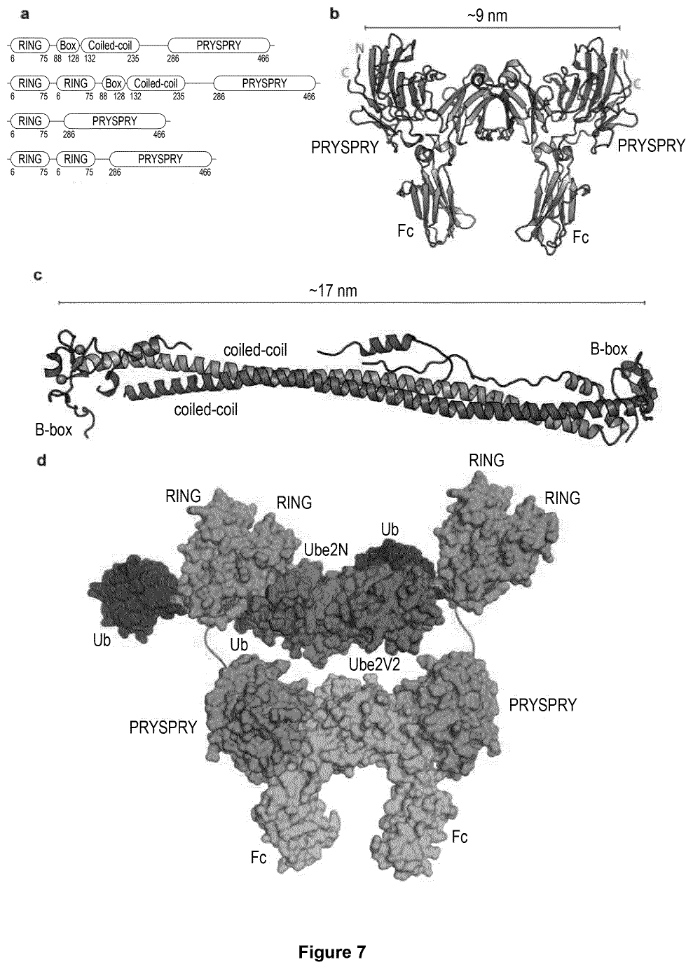

Finally, we considered whether the catalytic RING topology is an arrangement specific to Ube2N or one that also works with other E2 enzymes. Thus, we tested whether addition of Fc could induce self-ubiquitination of Ub-TRIM21 in presence of Ube2D1, a highly promiscuous E2 enzyme. However, even after extended reaction times hardly any TRIM21 modification was detected, while in contrast free ubiquitin chains could be observed (FIG. 8). The catalytic RING topology we observe in our structure is thus specific for Ube2N/Ube2V2, explaining why this enzyme and not Ube2D1 is required for TRIM21's cellular function. Moreover, this may explain why TRIM21, and other TRIMs such as TRIM5, build K63- and not K48-linked ubiquitin chains when first activated. Their mechanism of activation, induction of the catalytic RING topology, only results in formation of self-anchored K63 chains by using Ube2N/Ube2V2. Collectively, these data identify formation of a catalytic trans RING topology as the driving force behind self-ubiquitination of TRIM21 with Ube2N/Ube2V2.

### Example 4: Catalytic RING Topology Drives Targeted Protein Degradation

### Materials & Methods

In vitro transcription and RNA purification: For in vitro transcription of mRNA, constructs were cloned into pGEMHE vectors (Liman, E. R., Tytgat, J. & Hess, P. Subunit stoichiometry of a mammalian K+ channel determined by construction of multimeric cDNAs. Neuron 9, 861-871, (1992)). Plasmids were linearized using Ascl. Capped (but not polyA-tailed) mRNA was synthesized with T7 polymerase using the HiScribe™ T7 ARCA mRNA Kit (New England Biolabs) according to the manufacturer's instructions. The sequences of the purified protein/expressed mRNA are provided in SEQ ID Nos: 6-38.

Cell lines: NIH3T3-Caveolin-1-EGFP (Shvets, E., Bitsikas, V., Howard, G., Hansen, C. G. & Nichols, B. J. Dynamic caveolae exclude bulk membrane proteins and are required for sorting of excess glycosphingolipids. Nat Commun 6, 6867, (2015)) were cultured in DMEM medium (Gibco; 31966021) supplemented with 10% Calf Serum and penicillin-streptomycin. RPE-1 cells (ATCC) were cultured in DMEM/F-12 medium (Gibco; 10565018) supplemented with 10% Calf Serum and penicillin-streptomycin.

All cells were grown at 37° C. in a 5% CO₂ humidified atmosphere and regularly checked to be mycoplasma-free. The sex of NIH3T3 cells is male. The sex of RPE-1 cells is female. Following electroporation, cells were grown in medium supplemented with 10% Calf Serum without antibiotics.

For live imaging with the IncuCyte (Sartorius), cell culture medium was replaced with Fluorobrite (Gibco; A1896701) supplemented with 10% Calf Serum and GlutaMAX (Gibco; 35050061).

RPE-1 TRIM21 knockout cells were generated using the Alt-R CRISPR-Cas9 system from Integrated DNA technologies (IDT) with a custom-designed crRNA sequence (ATGCTCACAGGCTCCACGAA) (SEQ ID NO: 39). Guide RNA in the form of crRNA-tracrRNA duplex was assembled with recombinant Cas9 protein (IDT #1081060) and electroporated into RPE-1 cells together with Alt-R Cas9 Electroporation Enhancer (IDT #1075915). Two days post-electroporation cells were plated one cell per well in 96 well plates and single cell clones screened by western blotting for TRIM21 protein. A single clone was chosen that contained no detectable TRIM21 protein and confirmed TRIM21 knockout phenotype in a Trim-Away assay.

For the proteasome inhibition experiments MG132 (Sigma; C2211) was used ata final concentration of 25 μM.

Transient protein expression from mRNA: To enable precise control of protein expression levels, constructs were expressed from in vitro transcribed mRNA. mRNA was delivered into cells by electroporation using the Neon Transfection system (Invitrogen). For each electroporation reaction 8×10⁵ RPE-1 TRIM21-knock out or NIH3T3-Caveolin1-EGFP cells suspended in 10.5 μl of Resuspension Buffer R were mixed with 2 μL of the indicated mRNA in water. After electroporation, cells were transferred into antibiotic-free DMEM or DMEM/F-12 media supplemented with 10% FBS and left to incubate for 5 h before cells were harvested. Typically, expression could be detected from 30 min after electroporation and lasted for about 24 h.

Trim-Away: For each electroporation reaction 8×10⁵ NIH 3T3 Cav1-knock in cells suspended in 10.5 μl of Resuspension Buffer R were mixed with the indicated amount of antibody-mixture diluted in 2 μl of PBS. mRNAs were added immediately prior to electroporation, to limit degradation by potential RNAse activity. Cav1-GFP mRNA encoding Vhh-Fc (WT or PRYSPRY binding deficient H433A mutant) and TRIM21 were electroporated. The cell mRNA mixtures were taken up into 10 μl Neon electroporation pipette tips (Invitrogen) and electroporated using the following settings: 1400 V, 20 ms, 2 pulses (as described in Clift, D. et al. A Method for the Acute and Rapid Degradation of Endogenous Proteins. Cell 171, 1692-1706 e1618, (2017) and Clift, D., So, C., McEwan, W. A., James, L. C. & Schuh, M. Acute and rapid degradation of endogenous proteins by Trim-Away. Nat Protoc 13, 2149-2175, (2018)). Electroporated cells were transferred to antibiotic-free Fluorobright media supplemented with 10% FBS and left to incubate for 5 h in an incubator before the cells were harvested for immunoblotting. GFP-fluorescence measured using an Incucyte® (essenbioscience) and was normalized to the control (Vhh-Fc^{H433A}). Protein detection was performed using the following antibodies: Fc: goat antihlgG Fc broad 5211-8004 (1:2,000); TRIM21: rabbit anti-TRIM21 D101D (ST #9204) (1:1,000), Vinculin: rabbit anti-Vinculin EPR8185 ab 217171 (1:50,000); Caveolin-1: rabbit anti-Cav1 (BD: 610059, 1:1,000).

mEGFP-Fc degradation assay: For mEGFP-Fc degradation assay, 0.4 μM mEGFP-Fc mRNA together with 1.2 μM of the indicated TRIM21 mRNA were electroporated into 8×10⁵ cells, as described above. Electroporated cells were transferred to antibiotic-free DMEM supplemented with 10% FBS. For western analysis only, cells were incubated for 5 h in an incubator before harvest. For Flow cytometry analysis, the half of the cells were taken and treated with 25 μM MG132 while the other half were treated with DMSO. Then cells were incubated for 5 h in an incubator before being harvested. Cells were fixed before being subjected to flow cytometry. The same antibodies were used as for Trim-Away (see above).

Flow Cytometry: Cells were fixed prior to flow cytometry. For this, cells were resuspended in FACS fixative (4% formaldehyde, 2 mM EDTA in PBS) and incubated at room temperature for 30 min. Afterwards, cells were centrifuged and resuspended in FACS buffer (2% FBS, 5 mM EDTA in PSB) and stored at 4° C., wrapped in aluminium foil until use. Flow cytometry was performed using an Eclipse (iCyt) A02-0058. Cells were measured using forward and side scattering to assess live cells. In addition, green fluorescence was measured. Live cells were selected based on forward and side scattering and only the median GFP fluorescence of live cells was used for further analysis.

### Results

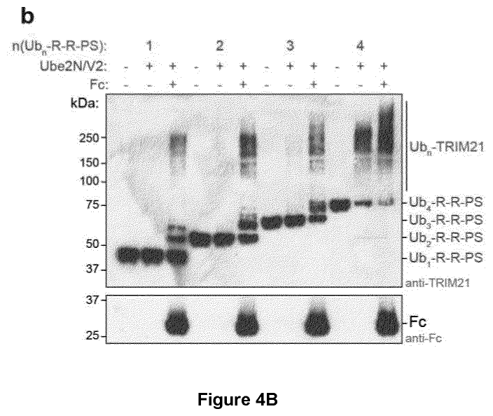

Having established the RING topology necessary for self-anchored ubiquitination in vitro, we next investigated if this same arrangement is required for TRIM21 activity in cells. We designed a similar series of TRIM21 constructs for cellular expression as above, which control for the number of RINGs available and their distance to each other when bound to Fc (FIG. 5a). We expressed these constructs in TRIM21 knock out RPE-1 cells together with GFP-tagged Fc and monitored GFP-Fc degradation as a readout for TRIM21 activity, in a targeted protein degradation experiment. Consistent with the inability to form anchored chains when engaged with Fc in vitro, full-length TRIM21 did not degrade GFP-Fc in cells (FIG. 5b, c). Degradation could not be rescued by addition of another RING to the N-terminus, presumably because in this case the RINGs are dimeric but still separated by the coiled coil, with the consequence that no ‘substrate’ RING is available for ubiquitination. Thus, RING dimerization is not sufficient for cellular TRIM21 activity. In the R-PS construct, the RINGs are within ˜9 nm, and thus within the range compatible with activity as defined by our structure (FIG. 3a). Despite this, no degradation was observed (FIG. 5b, c), likely because the RINGs can either form a single dimer, or one monomer RING would have to act as the enzyme and the other RING as the substrate. This is consistent with the inefficient self-ubiquitination of a comparable construct in our biochemical experiments (FIG. 3d). Only R-R-PS showed efficient GFP-Fc degradation (FIG. 5b, 5c). When this construct engages Fc, two RING dimers can form in close proximity, so that one RING dimer is available to mediate the ubiquitination of the other, thus fully satisfying the requirements of the catalytic RING topology. All the constructs were expressed at comparable levels and were active in classical Trim-Away targeted protein degradation assays (FIG. 5d, 5e), suggesting that the only difference is the number and relative distance of RING domains when engaged with the GFP-Fc construct. This also agrees with our biochemical data, where a similar construct shows strong self-ubiquitination upon substrate binding (FIG. 3d). Therefore, the Fc-induced self-ubiquitination assay in vitro provides a good prediction for cellular activity. Our crystal structure of the initiation of RING-anchored ubiquitin chain elongation therefore precisely visualizes how this process can work in a physiological context.

### Example 5

The catalytic RING topology we describe is consistent with data showing that TRIM proteins can undergo higher-order assembly, and that the present invention is not only applicable to TRIM 21 derived RING domains and RING E3 ligases derived from other polypeptides are also suitable RING domains for inclusion in the fusion proteins.

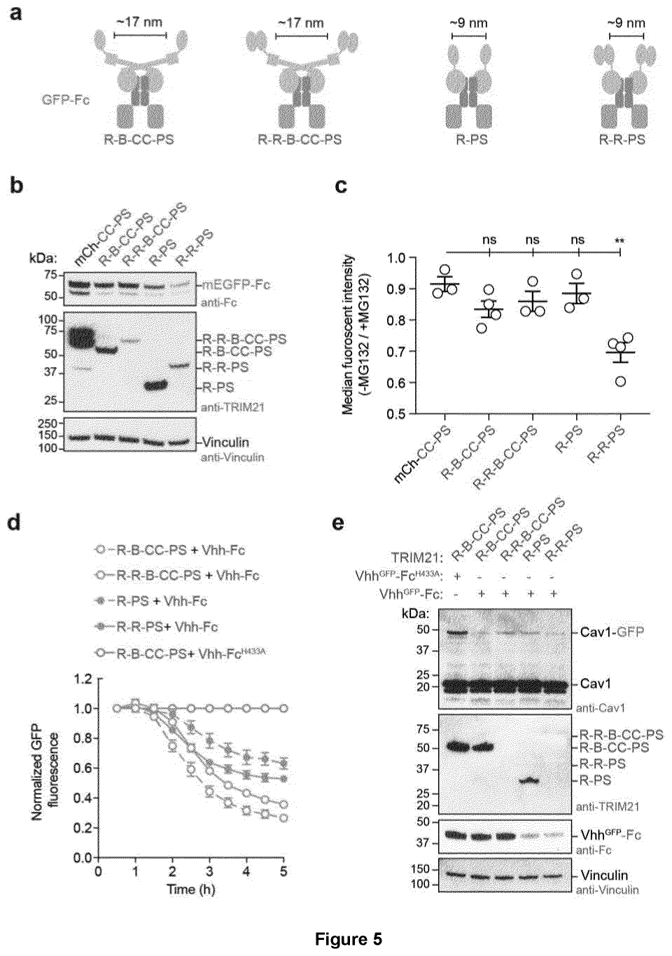

In the case of TRIM5α, three TRIM5α RINGs are brought into close proximity when the protein is incubated with the HIV capsid (Ganser-Pornillos, B. K. et al. Hexagonal assembly of a restricting TRIM5alpha protein. Proc Natl Acad Sci USA 108, 534-539, (2011), Wagner, J. M. et al. Mechanism of B-box 2 domain-mediated higher-order assembly of the retroviral restriction factor TRIM5alpha. Elife 5, 16309 (2016), Li, Y. L. et al. Primate TRIM5 proteins form hexagonal nets on HIV-1 capsids. Elife 5, 16269 (2016)) (FIG. 6a, b,). This positioning would fulfil the catalytic RING topology we describe and would be consistent with the ability of TRIM5α to restrict retroviruses and activate the innate immune response via self-anchored K63-ubiquitination (Stremlau, M. et al. The cytoplasmic body component TRIM5alpha restricts HIV-1 infection in Old World monkeys. Nature 427, 848-853, (2004), Sayah, D. M., Sokolskaja, E., Berthoux, L. & Luban, J. Cyclophilin A retrotransposition into TRIM5 explains owl monkey resistance to HIV-1. Nature 430, 569-573, (2004), Fletcher, A. J. et al. TRIM5alpha requires Ube2W to anchor Lys63-linked ubiquitin chains and restrict reverse transcription. EMBO J 34, 2078-2095, (2015), Pertel, T. et al. TRIM5 is an innate immune sensor for the retrovirus capsid lattice. Nature 472, 361-365, (2011)). The functional requirement for multiple TRIM molecules is also suggested by the fact that potent antibody-mediated neutralization of adenovirus by TRIM21 requires multiple antibodies bound per virus (McEwan, W. A. et al. Regulation of virus neutralization and the persistent fraction by TRIM21. J Virol 86, 8482-8491, (2012)). In addition, TRIM21 was shown to be activated by substrate-induced clustering, resulting in multiple TRIM21:antibody complexes on the substrate (Zeng, J. et al. Substrate-induced clustering activates Trim-Away of pathogens and proteins. doi: Pre-print at https://doi.org/10.1101/2020.07.28.225359 (2020), and now published as Zeng et al (2021) Natural Structural & Molecular Biology vol 28, 278-289). The unique TRIM-architecture, in which the RINGs are located at either end of a coiled-coil, and the flexibility provided by the hinge region of the antibody, may be crucial in enabling TRIM21 molecules bound onto the surface of a virus to engage with each other (FIG. 6c). To fulfil the catalytic RING topology on the virus, two RINGs need to dimerize and a third has to be within ˜9 nm of the RING dimer, enabling self-anchored ubiquitination and subsequent virus neutralization (FIG. 6d). Since higher-order assembly has been associated with many other K63 ubiquitin chain forming RING E3 ligases, such as TRAF6 (Napetschnig, J. & Wu, H. Molecular basis of NF-kappaB signaling. Annu Rev Biophys 42, 443-468, (2013)), RIPLET (Cadena, C. et al. Ubiquitin-Dependent and -Independent Roles of E3 Ligase RIPLET in Innate Immunity. Cell 177, 1187-1200 e1116, (2019)) and others, we propose that the mechanism presented here is thus likely to be found more widely within the realm of RING E3 ligases.

### Example 6

Protein degradation of endogenous target proteins (including the kinases IKK and Erk1) was assessed using different TRIM constructs, wherein R is a TRIM21 RING domain, PS is the PRYSPRY antibody binding domain of TRIM21, CC is a Coiled-Coil domain, B is a B-Box domain, T21 is full length TRIM.

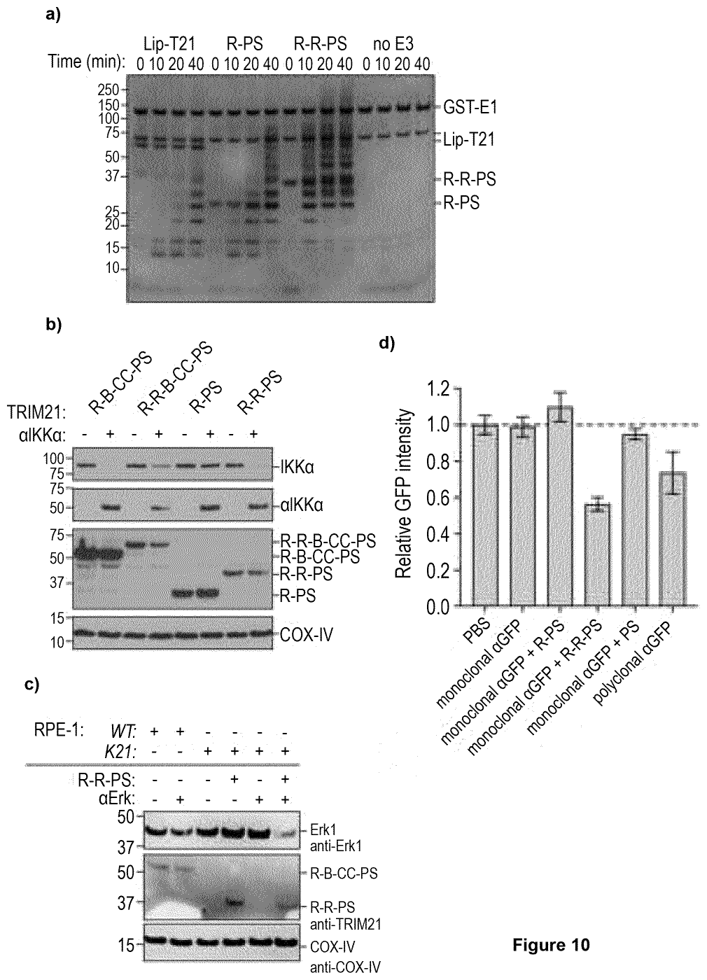

A) Catalysis of unanchored ubiquitin chains by Ube2N/Ube2V2 of different TRIM21 constructs (Lip-T21, R-PS, R-R-PS) at 10 μM concentration was performed. Ubiquitination assay was performed as described in (Kiss et al. (2021) Nature Communications, vol 12(1):1220). Shown in FIG. 10a is an InstantBlue gel of the reactions after 60 min.

B) Trim-Away of endogenous IKKα in RPE1 TRIM21 knock-out cells using transiently expressed TRIM21 constructs (R-R-B-CC-PS, R-B-CC-PS, R-R-PS and R-PS). 1.2 μM of mRNA encoding the respective TRIM21 construct were mixed with 140 ng rabbit αIKK IgG (Abcam, ab169743) in a volume of 2 μL. This electroporation mix was then added to 10.5 μL containing 8×10⁵ RPE-1 TRIM21 knock-out. The cell:mRNA:IgG mixture was taken up into 10 μL Neon electroporation pipette tips (Invitrogen) and electroporated using the following settings: 1400 V, 20 ms, 2 pulse (Neon Electroporator). Electroporated cells were transferred to antibiotic-free Fluorobright media supplemented with 10% FBS and left to incubate for 5 h in an incubator before the cells were harvested for immunoblotting. The results are shown in FIG. 10b

C) Trim-Away of endogenous Erk1 kinase in either RPE1 WT or TRIM21 knock-out cells using R-R-PS protein at 2.4 μM and αErk1 antibody at 0.5 μM concentration in the electroporation reaction. Electroporation was performed as described in above in B). Cells were harvested for western blot analysis after 1 h. Endogenous TRIM21 in RPE-1 cells would usually take 3-4 h for efficient Trim-Away of Erk1.

D) Trim-Away of ectopically expressed monomeric EGFP in RPE1 cells using mono- or poly-clonal antibody against GFP (0.5 μM) and different TRIM21 constructs (2.4 μM). Electroporation was performed as described above in B). GFP-fluorescence was measured using an Incucyte® (essenbioscience). Shown in FIG. 10d is the relative GFP intensity after 4.5 h.

### Results

The results in FIG. 10 show that the RR construct (i.e. constructs comprising two RING domains) was more efficient in degradation of the target protein compared to the single RING construct.

### Example 7

### Materials & Methods

To further assess the abilities of the TRIM constructs as degraders, the degradation of an EGFP fusion protein (Caveolin-1-EGFP) by TRIM constructs was assessed.

RPE-1 cells stably expressing a reporter construct (Caveolin-1-mEGFP) were generated with lentiviral transduction and selected by GFP positive cell sorting. The cells were then electroporated with a mix of monoclonal mouse anti-GFP antibody 9F9.F9 and the indicated TRIM21 purified protein constructs at a final electroporation tip concentration of 1 μM and 6 μM respectively. Immediately after electroporation cells were plated and Caveolin-1-mEGFP fluorescence monitored with the IncuCyte live cell imaging system. The data is normalised to total cell area and PBS control. At 3 h post-electroporation cells were lysed in RIPA buffer and lysates probed with anti-PRYSPRY (D1O1D) and anti-mouse IgG antibodies using the Jess simple western system (Biotechne).

His-Lipoyl-T21 is full length TRIM21. T21R-PRYSPRY and T21R-R-PRYSPRY are one TRIM21 RING domain (T21R-) or two RINGs (T21R-R-) fused to the PRYSPRY antibody binding domain of TRIM21. His-PRYSPRY is the PRYSPRY domain alone.

### Results

The anti-GFP antibody binds to Caveolin-1-mEGFP and recruits either endogenous cellular TRIM21 (anti-GFP) or the exogenous TRIM21 proteins (His-Lipoyl-T21, T21R-PRYSPRY, T21R-R-PRYSPRY and His-PRYSPRY) which are co-electroporated in 6-fold excess with anti-GFP antibody.

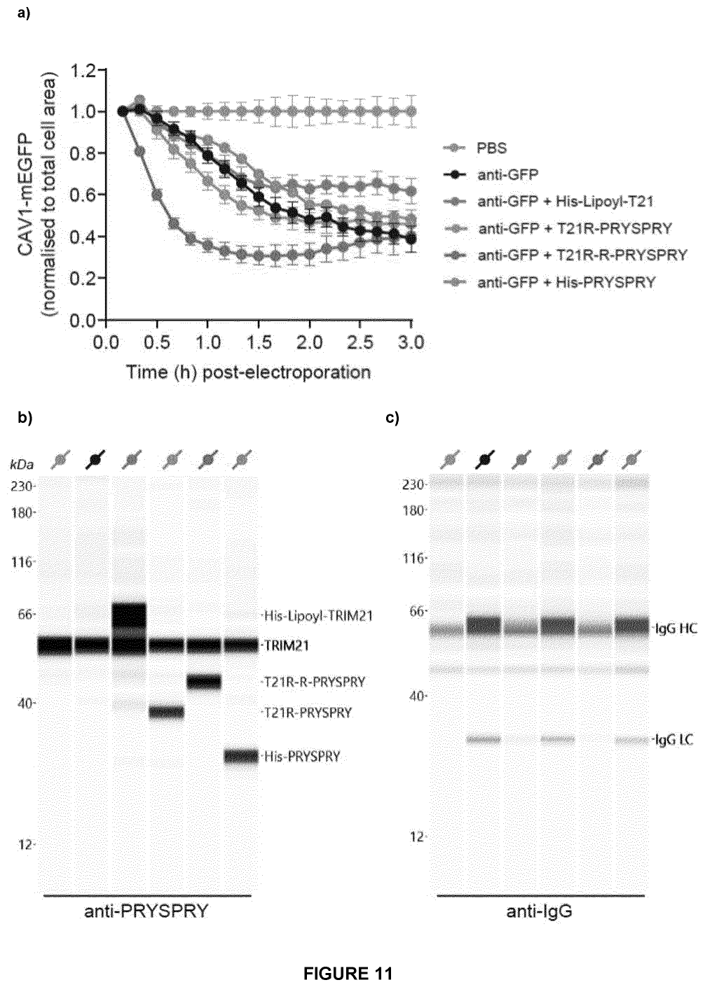

The T21R-R-construct drives faster and more efficient degradation. Degradation of the Caveolin-1-EGFP is monitored using real-time fluorescence microscopy (FIG. 11a). Western blots show levels of endogenous and exogenous electroporated TRIM21, as well as electroporated anti-GFP antibody (IgG-HC and IgG-LC) at the end of the experiment (FIGS. 11b and 11c).

These results show that the RR construct (i.e. two RING domain construct) is a faster and more efficient degrader of the target protein than the single RING construct.

### Example 8

To further assess the ability of the TRIM constructs as degraders, the degradation of an EGFP fusion protein (H2B-1-EGFP) by various TRIM constructs was assessed.

Various TRIM21 RING domain constructs fused to the anti-GFP nanobody vhhGFP4 by a flexible linker were generated using a combination of custom synthesis, PCR and Gibson assembly into a custom pOPT vector for bacterial expression in E. coli. Constructs T21R, T21RR and T21R(Dead—M72E, I18R) were expressed as fusion proteins with hexahistidine-SUMO tag at the N terminus, which was cleaved during purification resulting in an unmodified N terminus of Trim21. UbT21RR was expressed with a C terminal hexahistidine tag, which was cleaved during purification. Proteins were purified using standard methods using affinity and size exclusion chromatography.

RPE-1 cells stably expressing a reporter construct (H2B-mEGFP) were generated with lentiviral transduction and selected by GFP positive cell sorting. The cells were then electroporated with the indicated T21R-vhhGFP4 purified protein constructs at a final electroporation tip concentration of 1.6 μM. Immediately after electroporation the cells were plated and H2B-mEGFP fluorescence monitored using the Incucyte live cell imaging system. The data is normalised to total cell area and buffer control.

T21R and T21R-T21R are one TRIM21 RING domain (T21R-) or two RINGs (T21R-R) fused to vhhGFP4. Ub-T21R-R is two RINGs fused to vhhGFP4 with an N-terminal ubiquitin domain. T21R(dead) is one TRIM21 RING with I18R and M72E point mutations fused to vhhGFP4. This construct with a mutant RING domain is unable to dimerise or bind ubiquitin and therefore is catalytically inactive.

### Results

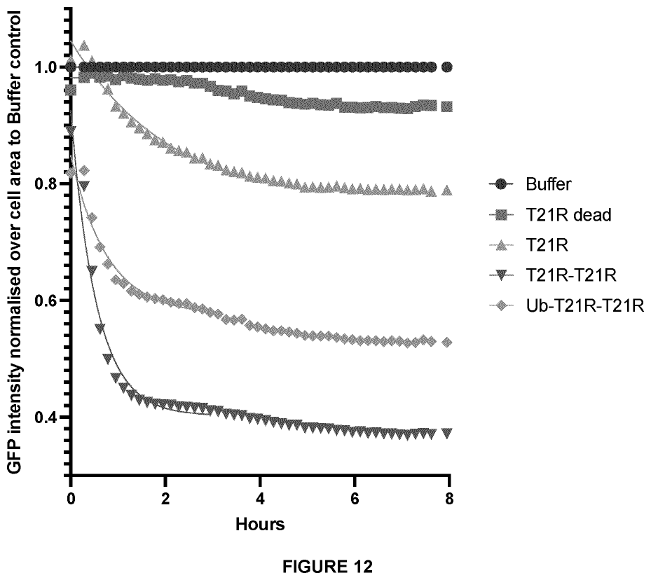

The various TRIM21 RING constructs are recruited to H2B-mEGFP via the anti-GFP nanobody. Degradation of the H2B-EGFP is monitored using real-time fluorescence microscopy (FIG. 12). The T21R-R-construct drives faster and more efficient degradation.

These results show that the RR construct (i.e. two RING domain construct) is a faster and more efficient degrader of the target protein than the single RING construct. These results show that a RING-RING fusion protein can be fused to a nanobody targeting domain, resulting in a more active degrader than when using a single RING construct. This suggests a dual RING fusion therapeutic could be superior to a single RING fusion therapeutic.

### Summary

A fusion protein has been developed comprising two RING E3 ligase domains, and a protein targeting domain. The results from these experiments support that such a fusion protein is capable of targeted protein degradation in a physiological setting. Therefore, such fusion proteins and nucleic acid constructs encoding the same are suitable for use in for the degradation of proteins in a cell in both a therapeutic and research application.

Here we provide a structural framework for understanding RING E3-anchored ubiquitin chain formation. We were able to capture a snapshot of this process in a crystal structure of mono-ubiquitinated TRIM21 RING (Ub-R) with the ubiquitin charged heterodimeric E2 enzyme Ube2N˜Ub/Ube2V2 (FIG. 1), showing the chemical activation of the acceptor ubiquitin, exemplified by the deprotonation of the acceptor lysine by Ube2N D119 (FIG. 2). Most importantly, our structure reveals the domain arrangement required for the elongation reaction, in other words a catalytic RING E3 topology that enables the extension of a mono-ubiquitinated RING into a K63-linked, RING-anchored ubiquitin chain (FIG. 3, 4). In this arrangement, two RINGs form a dimer and act as an enzyme on a third RING domain, which acts as the substrate in this reaction. We observe that while rigidity is required to position all the important catalytic residues in the E2 active site optimally (FIG. 2), formation of the substrate anchored ubiquitin chain likely requires conformational flexibility between domains that is provided by the unique topology of TRIM proteins (FIG. 3). Substrate-induced self-ubiquitination of TRIM21 is highly efficient, even at low ligase concentration, in contrast to free-ubiquitin chain formation (FIG. 3). This implies that physiological ubiquitin signals may not be produced as free chains but mainly on substrates, due to the higher reaction efficiency.

Our data establishes that the RING-anchored K63-chain is first formed in a trans-mechanism, where a RING dimer activates a Ube2N˜Ub molecule, thereby acting as an E3 ligase. An additional mono-ubiquitinated RING acts as a substrate for ubiquitination and accepts the donor ubiquitin (FIG. 3). Only after four ubiquitin molecules have been added to the RING in trans, is the chain sufficiently long for ubiquitin chain formation in cis (FIG. 4). While ubiquitin chain elongation in cis occurs at much higher rates, the initial need for a trans arrangement may represent an important regulatory mechanism suppressing TRIM21 activity in absence of a substrate. In the case of TRIM21 or TRIM5α, activation is driven by substrate binding, which is needed for trans ubiquitination. Interestingly, substrate modification with linear ubiquitin chains by the RBR (RING-in between-RING) ligase HOIP is regulated by its partner RBR HOIL, which mono-ubiquitinates all three LUBAC components HOIP, HOIL and SHARPIN. These ubiquitin primers are then elongated in cis by HOIP, thereby outcompeting trans ubiquitination of substrates (Fuseya, Y. et al. The HOIL-1L ligase modulates immune signalling and cell death via monoubiquitination of LUBAC. Nat Cell Biol 22, 663-673, (2020)). Thus, switching between cis and trans mechanisms of ubiquitination may be a regulatory system exploited by many different types of E3 ligases.

The catalytic RING topology observed in our structure predicts the requirements for TRIM21-mediated targeted protein degradation in cells (FIG. 5). Upon substrate recognition, TRIM21 forms a K63-linked ubiquitin chain on its N-terminus (Fletcher, A. J., Mallery, D. L., Watkinson, R. E., Dickson, C. F. & James, L. C. Sequential ubiquitination and deubiquitination enzymes synchronize the dual sensor and effector functions of TRIM21. Proc Natl Acad Sci USA 112, 10014-10019, (2015)). Loss of this K63-linked ubiquitin chain prevents virus neutralization, immune signalling and Trim-Away (Kiss, L. et al. A tri-ionic anchor mechanism drives Ube2N-specific recruitment and K63-chain ubiquitination in TRIM ligases. Nat Commun 10, 4502, (2019)). Our GFP-Fc degradation experiment shows that only the TRIM21 construct (R-R-PS) that can form the catalytic RING topology under these conditions enables degradation (FIG. 5). Interestingly, specific orientation of the E3 ligase CRL^{VHL} relative to its substrate was also shown to be critical for targeted protein degradation, (B. E. et al. Differential PROTAC substrate specificity dictated by orientation of recruited E3 ligase. Nat Commun 10, 131, (2019)).

The data also establishes that the TRIM constructs comprising two RING domains were more efficient degraders of the target protein than constructs comprising one RING domain (FIGS. 10-12)

1. A fusion protein comprising:
a first RING domain;
a second RING domain; and
a protein targeting domain.

2. A fusion protein according to claim 1 wherein the fusion protein does not comprise a domain selected from a coiled-coil domain and a B-box domain.

3. A fusion protein according to claim 1 or claim 2 wherein the fusion protein does not comprise a coiled-coil domain and does not comprise a B-box domain.

4. A fusion protein according to any one of claims 1 to 3 wherein the protein targeting domain is located at the C-terminal domain end of the first and second RING domains.

5. The fusion protein according to any one of claims 1 to 4 wherein the first RING domain and second RING domain are derived from TRIM polypeptides.

6. The fusion protein according to claim 5 wherein the TRIM polypeptides is selected from the group consisting of TRIM5, TRIM7, TRIM19, TRIM21, TRIM25, TRIM28 and TRIM 32.

7. The fusion protein according to claim 6 wherein the first RING domain and second RING domain are derived from the same TRIM polypeptide, preferably from TRIM21.

8. The fusion protein according to any one of claims 1 to 7 wherein the fusion protein comprises two RING domains.

9. The fusion protein according to any one of claims 1 to 8 wherein the protein targeting domain is a PRYSPRY domain, an antibody or antibody fragment thereof, or antibody mimetic, wherein the antibody fragment is preferably selected from the group consisting of a Fab, Fab′, F(ab′)2, scFab, Fv, scFV, dAB, VL fragments thereof, VH fragments thereof and V_{HH} fragments thereof.

10. The fusion protein according to any one of claims 1 to 9 further comprising linker sequences between the first and second RING domains and/or the second RING domain and the protein targeting sequence.

11. A nucleic acid construct encoding the fusion protein according to any one of claims 1 to 10.

12. A nucleic acid construct comprising a first nucleic acid sequence encoding a first RING domain, a second nucleic acid sequence encoding a second RING domain, and a third nucleic acid sequence encoding a protein targeting domain.

13. A nucleic acid construct according to claim 12, wherein the construct does not encode for a coiled-coil domain; does not encode for or a B-Box domain or does not encode for a coiled-coil domain and a B-box domain.

14. The nucleic acid construct according to any of claims 11 to 13 in the form of a vector.

15. The nucleic acid construct according to claim 14 wherein the vector is viral delivery vector, preferably an adeno-associated virus (AAV) vector.

16. A pharmaceutical composition comprising a fusion protein according to any one of claims 1 to 10 or a nucleic acid according to any one of claims 11-15, and a pharmaceutically acceptable carrier and/or excipient.

17. A method of treating a neurological disorder, an infection or a trinucleotide repeat disorder comprising administering a fusion protein according to any one of claims 1 to 10 or a nucleic acid according to any one of claims 11 to 15 to a subject.

18. The method according to claim 17 further comprising administering simultaneously or sequentially in any order, an antibody or antibody fragment thereof, or a nucleic acid construct encoding the same.

19. A fusion protein according to anyone of claims 1 to 10 or a nucleic construct according to any one of claims 11 to 15 for use as a medicament.

20. A fusion protein for use according to claim 19 for treating a neurological disorder, preferably the neurological disorder is Alzheimer's Disease or Huntington's Disease, for treating a viral infection, preferably an HIV infection, or for treating a trinucleotide repeat disorder.

21. A method of degrading a protein in a cell comprising introducing a fusion protein of any one of claims 1 to 10 or a nucleic construct according to any one of claims 11 to 15 into the cell.

22. A method according to claim 21 wherein introducing the fusion protein or nucleic acid into the cell is carried out by transfection or transduction, preferably by using a vector, injection or electroporation.

23. A method of degrading a protein in a sample comprising introducing a fusion protein of any one of claims 1 to 10 or a nucleic construct according to any one of claims 11 to 15 into the sample.

24. A method according to any one of claims 21 to 23 further comprising introducing an antibody or antibody fragment thereof or a nucleic acid encoding the same into the cell or sample.
